(19) United States
(12) Patent Application Publication
(10) Pub. No.: US 2017/0094920 A1
(43) Pub. Date: Apr. 6, 2017

(54) INTEGRATED INCUBATION, CULTIVATION AND CURING SYSTEM AND CONTROLS FOR OPTIMIZING AND ENHANCING PLANT GROWTH, DEVELOPMENT AND PERFORMANCE OF PLANT-BASED MEDICAL THERAPIES

(71) Applicants: Craig Ellins, Las Vegas, NV (US); Todd Denkin, Henderson, NV (US); Wayne Love, Henderson, NV (US); Ulrich Reimann-Philipp, Las Vegas, NV (US); Andrea Small-Howard, Los Angeles, CA (US); Lucas Marin, Las Vegas, NV (US); Jorge Velez, San Juan, PR (US); Cesar Cordero Kruger, Santurce, PR (US); Long Nguyen, Las Vegas, NV (US)

(72) Inventors: Craig Ellins, Las Vegas, NV (US); Todd Denkin, Henderson, NV (US); Wayne Love, Henderson, NV (US); Ulrich Reimann-Philipp, Las Vegas, NV (US); Andrea Small-Howard, Los Angeles, CA (US); Lucas Marin, Las Vegas, NV (US); Jorge Velez, San Juan, PR (US); Cesar Cordero Kruger, Santurce, PR (US); Long Nguyen, Las Vegas, NV (US)

(21) Appl. No.: 14/873,413

(22) Filed: Oct. 2, 2015

(51) Int. Cl. A01G 31/02; A01G 7/04

(52) U.S. Cl. CPC A01G 31/02; A01G 7/045

(57) ABSTRACT

An integrated incubation, cultivation and curing system and controls for optimizing, standardizing and enhancing plant-based medical therapies by controlling and regulating plant growth, development and performance at any stage of a plant's development including propagating, growth, flowering, fruit formation or during processes associated with the handling of the culture through multiple automated, enclosed and controlled environmental systems and thereby standardizing the resultant product.

### CROSS REFERENCES TO RELATED APPLICATIONS

Not applicable. The present application is an original and first-filed United States Utility patent application.

### STATEMENT REGARDING FEDERALLY SPONSORED RESEARCH OR DEVELOPMENT

Not applicable.

### THE NAMES OR PARTIES TO A JOINT RESEARCH AGREEMENT

Not applicable.

### INCORPORATION BY REFERENCE OF MATERIAL SUBMITTED ON A COMPACT DISC

Not applicable.

### BACKGROUND OF THE INVENTION

### 1. Field of the Invention

The present invention relates generally to an integrated incubation, cultivation and curing system and related controls for optimizing, standardizing and enhancing plant-based medical therapies by controlling and regulating plant growth, development and performance at any stage of a plant's development including propagating, growth, flowering, fruit formation or during processes associated with the handling of the culture through multiple automated, enclosed and controlled environmental systems and thereby standardizing the resultant product.

More particularly the invention relates to a multiplicity of enclosed chambers, each with growth stage determinant environmental controls and sensors, integrated to optimize, standardize and control a plant's growth cycle and development, each chamber having reflective interior wall elements, LED lighting at specified wavelengths and intensity, monitoring and control systems which may be employed among and between the various chambers to accelerate plant growth and provide a uniform, certifiable quality plant, particularly for medical therapies.

The invention further relates to a suite of enclosed chambers, each of which has dedicated and integrated growth optimization systems to provide a first stage sterile growth environment for tissue cultures and generating mini-clones, a second stage growth chamber for rooting mini-clones, a third stage growth chamber for growing plants to a size suitable for flowering, a fourth stage growth chamber to accelerate and optimize the growth of the flowering plants and a fifth stage chamber for uniformly and in a controlled manner curing the harvested flowers. Each of the growth chambers has a series of sensors and controls to provide optimal moisture, nutrients, carbon dioxide, LED light energy and related growth and performance optimizing agents to the plant at its respective stage in the growth cycle and to create a standardized, replicable uniform and quality certifiable plant, particularly for medical therapies, in an automated, controlled and contained manner.

The invention more particularly relates to a high efficiency, multi-chamber enclosed system with integrated controls systems within each chamber of the system and across the multiple chambers, to provide optimal incubation, growth, cultivation and curing for medicinal and non-medicinal plants in order to maximize their efficacy, enhance their growth, performance and development and create certifiable plants with replicated efficacy over time for each group of plants. The apparatus and system will be described in relationship to a system and related controls for standardizing, optimizing and providing certified plants. This is not to be understood as any limitation inasmuch as the system may be employed with a number of plants, growth optimization methodologies and different controls and sensors.

### 2. Plants in Medical Therapies

Medicinal plants have been identified and used throughout human history. Plants have the ability to synthesize a wide variety of chemical compounds that are used to perform important biological functions, and to defend against attack from predators such as insects, fungi and herbivorous mammals. At least 12,000 such compounds have been isolated so far; a number estimated to be less than 10% of the total. Chemical compounds in plants mediate their effect on the human body through processes identical to those already well understood for the chemical compounds in conventional drugs; thus herbal or botanical medicines do not differ greatly from conventional drugs in terms of how they work. This enables botanical medicines to be as effective as conventional medicines, but also gives them the same potential to cause harmful side effects.

The use of plants as medicines predates written human history. Ethnobotany (the study of traditional human uses of plants) is recognized as an effective way to discover future medicines. In 2001, researchers identified 122 compounds used in modern medicine which were derived from “ethnomedical” plant sources; 80% of these have had an ethnomedical use identical or related to the current use of the active elements of the plant. Many of the pharmaceuticals currently available to physicians have a long history of use as herbal remedies, including aspirin, digitalis, quinine, and opium.

Cannabis, more commonly known as marijuana, is a genus of flowering plants that includes at least three species, Cannabis sativa, Cannabis indica, and Cannabis ruderalis, as determined by plant phenotypes and secondary metabolite profiles. In practice however, cannabis nomenclature is often used incorrectly or interchangeably. Cannabis literature can be found referring to all cannabis varieties as “sativas” or all cannabinoid producing plants as “indican”. Indeed the promiscuous crosses of indoor cannabis breeding programs have made it difficult to distinguish varieties, with most cannabis being sold in the United States having features of both sativa and indica species.

The use of cannabis for social and medical purposes has been known for almost of all humanity's recorded history. Cannabis is most commonly administered via inhalation or consumption of marijuana-infused food and drink. However, since 1972 marijuana has been classified as a Schedule I drug under the U.S. Controlled Substances Act because the U.S. Federal Government considers it to have “no accepted medical use.” In stark contrast to this position, 23 of the 50 U.S. states and the District of Columbia have recognized the medical benefits of cannabis and have decriminalized its medical use.

President Obama has publicly commented on the recreational legalization of cannabis in Colorado and Washington stating that “it's important for it to go forward because it's important for society not to have a situation in which a large portion of people have at one time or another broken the law and only a select few get punished”. Indeed in the same interview, President Obama remarked about cannabis “I don't think it's more dangerous than alcohol. In fact, it is less dangerous than alcohol in terms of its impact on the individual consumer.” (Conor Friedersdorf January 2014, “Obama on Pot Legalization: ‘It's Important for it to go Forward’” The Atlantic). In line with the President's comments the U.S. Attorney General Eric Holder announced that the federal government would allow states to create a regime that would regulate and implement the legalization of cannabis, including loosening banking restrictions for cannabis dispensaries and growers (Jacob Sullum “Eric Holder Promises To Reassure Banks About Taking Marijuana Money ‘Very Soon’” Forbes January 2014).

In addition to these recent developments, the U.S. government has already set a precedent for patenting cannabis, and cannabis-related inventions. For example, U.S. Pat. No. 6,630,507 issued on Oct. 7, 2003 and assigned on the patent face to The United States of America, is directed to methods of treating diseases caused by oxidative stress by administering therapeutically effective amounts of a cannabidiol (CBD) cannabinoid from cannabis that has substantially no binding to the N-methyl-D-aspartate (NMDA) receptor, wherein the CBD acts as an antioxidant and neuroprotectant. A search of the U.S.P.T.O patent application Information Retrieval (PAIR) system also reveals the existence of thousands of cannabis related applications and issued patents including U.S. Pat. No. 8,034,843 (use of cannabinoids for treating nausea, vomiting, emesis, motion sickness), U.S. Pat. No. 7,698,594 (cannabinoid compositions for treatment of pain), and U.S. Pat. No. 8,632,825 (anti-tumoural effects of cannabinoid combinations) among many others. Thus, despite the official position of the U.S. Federal Government, and as recognized by the states that have legalized it, cannabis has been shown to provide substantial benefits for medical uses. Cannabis is regularly used by a wide cross-section of society to treat a variety of maladies, conditions and symptoms including, but not limited to, the following: nausea, glaucoma, lack of appetite, mucous membrane inflammation, epilepsy, leprosy, fever, obesity, asthma, urinary tract infections, coughing, anorexia associated with weight loss in AIDS patients, pain, and multiple sclerosis.

However, Cannabis intoxication (i.e., euphoria, relaxation) can occur and other side effects may also accompany its use, particularly with higher doses, specific cannabis varieties and/or over prolonged periods of usage. This is particularly true when there is little or no standardization of the resultant distributed product and no certification that the product is of a certain specified and repeatable efficacy. Undesirable side effects of using the available THC-predominant cannabis varieties can include, but are not limited to, the following: decreased short-term memory, dry mouth, impaired visual perception and motor skills, erectile dysfunction, lower fertility, red (i.e., blood shot) eyes, increased anxiety, occasional infarction, stroke, paranoia, acute psychosis, lowered mental aptitude, hallucinations, bizarre behavior, irrational panic attacks, irrational thoughts and various other cognitive and social problems.

Some of the negative or undesirable side effects from using available cannabis varieties for medical and recreational purposes are related to the plant's content of the chemical, DELTA 9-tetrahydrocannabinol (THC). A major hurdle to the more wide-spread acceptance of cannabis and its legalization is that the land races and commercially available cannabis genotypes (of drug varieties) contain relatively high concentrations of THC. Indeed the average THC content of traditional recreational cannabis has risen over the years from an average of 0.74 in 1975, to 3.35% in the 1990's, and average of 6.4% in 2003 (Annual Reports Nov. 9, 1999 to Nov. 8, 2003 of Mahmoud A. ElSohly, PhD, Director of the National Institute on Drug Abuse (NIDA) Marijuana Project at the National Center for Natural Products Research, School of Pharmacy, University of Mississippi). Recreational growers now report THC potency values as high as 30%.

There is a real need for cannabis and other medical plant varieties for potential medical use that have standardized and replicatable effectiveness, in order to produce THC concentrations or concentrations of other pharmacologically active substances that increase the medical benefits realized from their respective use. The inventions described herein meet that long-felt need and permit the growth of certified plants and the creation of related standardized plant-based medical therapies.

### 3. Need to Generate Medical Plant Uniformity

In order to ultimately have a plant-based therapy that meets regulatory norms, it is necessary to provide a plant with the desirable and favorable characteristics and profile that can be replicated indefinitely, hereby giving patients access to a plant-based medical product that repeatable efficacy at defined and established dosages.

Generally, most plant growers and particularly marijuana growers use a method called vegetative propagation (a.k.a. vegetative cloning), which is the act of cutting a large piece of a plant (generally a small branch with a number of leaves) and rooting the branch to produce a new plant. The resulting plant, referred to as a “clone” is genetically identical to the donor plant, which is affectionately referred to as the mother plant.

Growers use a process to select plants with favorable characteristics, such as, in the case of cannabis, a desirable cannabinoid or terpenoid. Vegetative propagation limits the number of plants that can be generated. In vegetative propagation plant quality is often inferior as using vegetative propagation can carry plant diseases onto the next generation. Thus it can decrease product quality and yield. Finally, the mother plants that donate their cuttings in the vegetative propagation process are not immortal. Growers may find a mother plant with highly desirable characteristics, but are unable to preserve its genetic properties indefinitely.

Thus it is necessary to be able to be able to provide standardized and “infinitely” repeatable plants with specific, certifiable qualities to ensure that a plant-based therapy has the maximum likelihood of success with minimum unexpected side effects due to plant variation. The inventions herein meet that need by using an advanced technique known as plant micro-propagation, which is performed under sterile tissue culture conditions, dramatically increases the yield of plantlets, and creates pluripotent plant propagation materials that may be cryogenically banked for future crops. The inventions described herein further meet that long-felt need and permit the initial creation of plant tissue to permit the ultimate growth of certified plants and the creation of related standardized plant-based medical therapies.

### 4. Plant Growth and Flowering

Plants can only take up nutrient ions that are located in the vicinity of the root surface. In nature, positioning of the nutrient ion can occur by one or more of three processes. The root can “bump into” the ion as it grows through the soil. This mechanism is called root interception. It is generally found that perhaps one to five percent of the nutrients in plants grown in soil come from the root interception process.

The soluble fraction of nutrients present in soil solution (water) and not held on the soil fractions flow to the root as water is taken up. This process is called mass flow. Nutrients such as nitrate-N, calcium and sulfur are normally supplied by mass flow.

Nutrients such as phosphorus and potassium adsorb strongly to soils and are only present in small quantities in the soil solution. These nutrients move to the root by diffusion. As uptake of these nutrients occurs at the root, the concentration in the soil solution in close proximity to the root decreases. This creates a gradient for the nutrient to diffuse through the soil solution from a zone of high concentration to the depleted solution adjacent to the root. Diffusion is responsible for the majority of the P, K and Zn moving to the root for uptake.

However, as can be appreciated from the above nutrient positioning mechanisms, uptake can be a fairly random event and result in non-optimal growth and development for a plant. The actual nutrient uptake process may cause a plant not to grow in an optimal manner.

Uptake of nutrients by a plant root is an active process. As water is taken up to support transpiration, nutrients may be moved to the root surface through mass flow. At this point, an active uptake process that requires energy is used to move the nutrients into the root cells and translocate them to the vascular system for transport to the growing tissues.

Specific protein carrier structures are used to bind nutrient ions and transport them across the root cell membrane. This active uptake process is also selective. The root cells discriminate and only expend energy to take up those nutrients the plant needs. Thus, nutrient uptake is not proportional to the ratios of nutrients in the solution. Ions in large supply in the solution, such as calcium and sulfur, can accumulate near the root. In perennial plants this can actually result in visible quantities of calcium carbonate and calcium sulfate precipitating and coating old roots.

One important implication of the plants ability to pick and choose nutrients from the solution is the relative unimportance of the ratio of nutrients in the solution. As long as a given nutrient is supplied to the root surface at a concentration high enough to meet the demands of nutrient uptake, the demands of growth and development will normally be met. For example, the ratio of calcium and magnesium on the cation exchange sites on the root and in the solution has little effect on the ratio of these nutrients in the plant. The plant selects the ions it needs, allowing the others to accumulate in the solution at the root surface. Altering the nutrient solution to supply adequate amounts, the concept of critical concentrations, has generally proven more cost effective than altering solutions to provide ratios of nutrients equivalent to the ratios at which the nutrients are found in the plants.

Thus, it would be desirable and advantageous to be able to supply a plant with the nutrients it needs in the amounts that it requires them, thus minimizing waste of nutrient supplies and optimizing a plant's ion selection action. This would also minimize excessive accumulation of unused nutrient salts at the root surface. It is also important to note that the normal patterns of nutrient uptake parallel plant vegetative growth in many ways. Most plants, and particularly crops that are to provide food or for medical usage take up the majority of the nutrients during the periods of vegetative growth and translocate stored nutrients to developing flowers, seeds and fruit during reproductive growth.

The amount and composition of the nutrient mix that the plant needs for optimal growth change during its development. Nutrient uptake increases rapidly from the early stages of growth to just prior to generation of reproductive mechanisms, and then stays at high levels until after pollination. Thus, it would be highly advantageous to be able to regulate the amount of nutrients available during respective growth phases and vary them according to the relative needs at any given time, thereby further minimizing nutrient waste.

### 5. Curing of Plant-Based Medicines

Plants are harvested when the flowers are ripe. Generally, ripeness for Cannabis is defined as when the white pistils start to turn brown, orange, etc. and start to withdraw back into the false seed pod. The seed pods swell with resins usually reserved for seed production, and we have ripe flowers with red and golden hairs.

Curing a cannabis harvest is an important process for anyone who wants to create the highest quality product with the maximum efficacy. Curing a vegetative crop involves not only drying the plant material, but also allowing for chemical conversions of the plant material and finally removing any fungal or microbial contaminants using a programmable heat/drying cycle. However, curing is often a time consuming process that is not regularly done by growers. A cannabis flower weighs more when fully ripe, and this is what most growers like to sell. Indeed, it is almost counterproductive for a grower who sells by weight and is not highly interested in either quality, optimization or the uniformity of the resultant product to cure the harvest since it ultimately results in a lower weight per volume, and, therefore, may actually reduce the amount received from a purchaser.

The curing process takes place after the drying process and allows for additional chemical conversions to happen that increase the quality of the flower. Firstly, it gives bacteria time to break down the remaining chlorophyll in the plant matter. Chlorophyll is the green pigment found in almost any plant and it is a vital component for photosynthesis—the means by which plants create food for themselves. However, chlorophyll contains magnesium which when burnt causes an undesirable quality to the final product. By curing the cannabis flower, one removes a lot of this, dramatically increasing the overall quality of the product and reducing its potential inefficiencies.

The second advantage of curing is that it allows further control of the moisture level of the flower material. Drying the flower material removes water, resulting in a stronger and easy to burn product. However, the drier the flower material gets the more it loses its taste and aroma due to loss of volatile terpenoid compounds. By curing just at the point when it is dry enough to burn, but not burn very well, one gains a finite level of control over just how much moisture is in the final botanical product as it finishes. However, without a control system, it is difficult to assess when that optimum point is. Thus, the current invention provides for optimum moisture-removal by implementing a controlled curing system and device to fill this need.

It is also important to note that the time of harvest controls the potency of the flower materials. If harvested “early” when only a few of the pistils have turned color, the flower materials will have a more pure THC content and will have less THC that has turned to CBD and CBN. The lessor psychoactive substances will create the bouquet of the botanical product, and control certain physiological and psychological responses by a user. Flower materials taken later, when fully ripened will normally have these higher CBN, CBD levels. All of this is an aspect of the control and optimization of the curing chambers, which is part of the instant invention.

When the trichomes (small hairs or other outgrowth from the epidermis of a plant, typically unicellular and glandular that exude an oily substance that contains THC and other chemicals) are mostly clear, not brown, the peak of floral bouquet is near. Once they are mostly all turning brownish in color, the THC levels are dropping and the flower is past optimum potency, and declining with light and wind exposure rapidly. Thus it is a further aspect of this invention to eliminate the deleterious effects of light and wind exposure by controlling the growth in a series of growth chambers and subsequent curing in a curing chamber, while trying to optimize the desired levels of active cannabinoid and terpenoid substances so as to create a uniform, repeatable and optimized plant-based medical product.

### 6. Multi-Purposed Integrated System to Provide Ecological Benefits

As the population on Earth increases and the improper development and usage of natural resources continues, arable lands disappear and vegetation on the earth's surface decreases at rapid rates. As a result, the problem of food shortage is getting more serious, the ability of converting carbon dioxide (CO₂) into oxygen (O₂) in the atmospheric environment by photosynthesis is reduced substantially, and the problem of global warming caused by greenhouse effect has gone from bad to worse. The need to maximize the use of arable lands for sustainable agriculture is sometimes outweighed by the desire to maximize the profitability of each arable acre with high-dollar yield crops that may have little or no nutritional value and may have deleterious health consequences, such as tobacco production.

Abnormal climatic changes are caused by the continuously increaseing temperatures on the earth's surface because of greenhouse effect. The climatic changes are the cause of: a) the yearly reduction of global rainfall and the reduction of accumulated snow on high mountains both of which result in the decline of water sources and droughts; b) the rise of sea level which results in flooding and the reduction of land area; the excessive rainfall in regional areas which results in the changes of growing cycles as well as distributions of plants and crops. As a result, plants and crops are seriously affected by floods, droughts, windstorms, plant diseases as well as insect pests. Thus it is imperative that water usage be optimized in plant cultivation and that methodologies be developed that permit water absorption and nutrient delivery to maximize a plant's growth and enhance its productivity.

Developing large areas of arable lands, improving cultivation techniques and adapting crop cultivars through selective breeding are time-consuming and alone cannot cope with the problems of food shortage and decline of arable land caused by droughts, floods, plant diseases, insect pests and chilling injury that are in part caused by climate change. Current agricultural practices including large scale genetic plant programs often create new or competing problems and issues. Moreover, improvement on the breeds of plants and crops is time-consuming. Furthermore, because arable lands on the Earth are limited, expanding the scale of cultivation is not feasible even if new breeds of plants and crops are developed successfully. Therefore, food shortage is still a problem which remains unsolved.

Many non-edible plants that have useful properties often need to compete for arable land with food crops. Medicinal plants have been cultivated and processed by individuals, families and communities from the beginning of humankind. Preparation methods for a myriad of medicinal use plants have been handed down, modified or lost over time. For many years, the cultivation, preparation, and use of certain medicinal plants was limited by cultural or religious concerns, or legally prohibited by governments.

In recent years, government restrictions on the cultivation, preparation, and/or use of certain medicinal plants have been revised or relaxed. As such, needs have arisen for controlled and optimized facilities in which medicinal plants can be cultivated and prepared for therapeutic or recreational uses. Ideally, the growth of these medicinal plants would take place under controlled and optimized conditions to create botanical materials, for distribution specifically to persons who are legally authorized or permitted to do so in certain countries, states or regions. In some situations, the quantity of a medicinal plant possessed by an individual is regulated.

Aeroponics, which is also called “air culture” or “soilless culture”, is presently the most modern and technologically evolved cultivation system for plant production. In aeroponics, plants are grown in the absence of any substrate. The nutrient solution is sprayed directly on the plant roots, which grow suspended in air within closed trays or vessels. The ideal conditions of absorbing oxygen, water and nutrient ions by the plants' root system result in the more rapid growth and maturation rates of the plants, the bigger density of planting and the easier control of pests and diseases. Also, plant cultivation can be repeated year-round without interruption.

Air culture systems available today around the world for research or for productions purposes, are closed cultivations systems, usually consisting of:

A central control unit (head tank), or peripheral units for managing parts of the system and containers for automatic preparation of nutrient solution by mixing nutrient stock solutions with automatic adjustment of pH and conductivity values.

An automatic irrigation system for spraying or misting the nutrient solution under low or high pressure onto the plants' roots, controlling the duration and frequency of spraying with automatic regulation of the time and frequency of injection. The nutrient solution is re-circulated from the plant growing trays or vessels back to the central control unit.

Trays or vessels into which the root system develops are arranged vertically or horizontally and are made from plastic or metal materials of different types, shapes and forms. In many cases, the container in which plants are grown also contains the nutrient solution.

The aeroponic systems which have been constructed so far have several major drawbacks, which have prevented their widespread application. One such drawback is that there has previously been no system to adjust the temperature to the optimal level for individual plants or groups of plants within one system. The temperature is a critical factor in relation to the type of crop plant and external temperature conditions. Also containers or channels into which development of the root systems occurs are not insulated properly. Plastic or metal materials are mainly used today for channels or receptacles into which the developed root systems are confined. These do not offer insulation.

A second major drawback of the currently known aeroponic cultivation systems is that they cannot simultaneously support multiple cultures of various plants (multicrop), or cultures with different nutritional needs. Similarly, currently known aeroponic systems do not provide optimal protection from outside contaminants such as air-borne and water-borne harmful chemicals, nor from infection and infestation by pathogens and pests. They also do not maximize the wavelength spectrum and photon flux of the available light, while simultaneously employing energy efficient technology to minimize the power consumption of the light source.

Traditional aeroponic fogging/hydroponic foggers have be used for many horticultural applications including root fogging, foliar feeding, growroom & greenhouse humidity generation and even ultra low volume (ULV) pesticide application. These ultrasonic foggers assist in propagation and production and can be used to optimize the environments for plants to grow. An aeroponic fogger can operate by oscillating at a frequency of approximately 2 MHz, which is two million vibrations per second. At this frequency, water is nebulized into a cold fog/dry fog that can support the needs of plants using an ultra low volume (ULV) of water and nutrients. An aeroponic fogger may also generate an extremely small droplet that averages only 2.5 microns which is small enough to be absorbed by roots and leaves on contact and can be effective using only an ultra low volume of liquid.

However, it has been determined that excessive fogging may have deleterious effects such as root rot. Regular fogging (5 μM droplets) is the likely cause of lower stem rot in certain aeroponic applications and by itself not sufficient to deliver all nutrients. An aspect of the invention is the unexpected discovery that intermittent spraying of the roots with a coarser mist (20-50 μM droplets) provides much better results. The fog is not essential for growing the plant.

Fog can still be useful for “shocking” roots in order to elicit biochemical responses, to adjust humidity in the root zone, and to deliver oxidizers or other chemicals to sanitize the roots.

It has also been discovered, and is part of this invention, that fog can be applied as an insurance in case the roots dry out or to deliver sudden stress.

Current aeroponic systems also do not employ “just-in-time” fogging or misting to provide the roots with just enough nutrient solution in a fine mist to provide the necessary nutrients for optimal growth while also providing growth stimulating oxygen at the optimal levels to maximize the plant's root development.

In addition, current aeroponic systems to not employ control feedback loops to simultaneously provide data on current crops to maximize yields and generate long term data to apply to analytical models that permit future plantings and harvests to be optimized both as to yield, quality and timing. The data and analytics permit successive crops to be planted and harvested to provide a substantially continuous yield with optimal harvest times in close proximity to one another while simultaneously not overstocking the market with product and causing an oversupply at a particular time.

Accordingly, the present invention, in addition to being used in medical plant generation, seeks to address one or more of the above-described situations and needs for other plants.

### SUMMARY OF THE INVENTION

It is therefore one object of the present invention to provide a method of enhancing the metabolic and growth processes and functions of plants by optimizing the growing conditions of these plants over the lifecycle of the plant.

It is still another object of the present invention to provide an integrated cultivation suite having a multiplicity of growing chambers and related equipment to facilitate the global manufacturing and distribution of cannabis and other plant-based medical solutions to combat a variety of clinically diagnosed health issues.

It is still another object of the present invention to provide an integrated cultivation suite having a multiplicity of growing chambers and related equipment to create safe, standardized pharmaceutical-grade cannabis-based therapies that target a variety of medical conditions.

It is therefore one object of the present invention to provide a self-contained growing unit having multiple growth chambers that isolates and protects plants growing inside while generating ideal growing environments to ensure high quality, purity, and consistency, both for medical plants and non-medical plants.

It is still another object of the present invention to provide an integrated cultivation suite that has as a part thereof a tissue culture incubator programmed to regulate lighting, temperature, and humidity to facilitate cell multiplication and the formation of embryos and shoots from sterile explants.

It is still another object of the present invention to provide an integrated cultivation suite that has as a part thereof a grow chamber for rooting mini-clones from a tissue culture incubator or for starting cutting from a mother plant.

It is yet another aspect of the present invention to provide an integrated cultivation suite that has as a part thereof a grow chamber for growing plants to a predetermined size in preparation for flowering.

It is still another object of the present invention to provide an integrated cultivation suite that has as a part thereof a grow chamber for flowering plants for enhancing the metabolic functions and the growing conditions of plants by optimizing the nutrient absorption and providing variable nutrient supplies based upon developmental stage, physiological responses, absorption rates and other variables for which the invention is able to obtain data to be used to model future plant growth enhancement.

It is yet another aspect of the invention to provide an integrated system for growing medicinal and recreational plants and non-medical plants comprising a tissue culture growth environment, a nursery growth environment, a growth environment in preparation for flowering and a growth environment through flowering, each growth environment comprising one or more enclosures, a support structure positioned in the grow environment enclosure and adapted to support growing medicinal or recreational plants and non-medical plants, sensors to monitor the growth variable factors including, but not limited to, temperature, light, humidity, carbon dioxide and water and nutrient delivery, an nutrient delivery system coupled to the support structure and adapted to deliver micro-droplets of nutrient-laden mist or dry fog to the medicinal or recreational plants and non-medical plants, a variable intensity and wavelength light system positioned in the grow environment enclosure and adapted for growing medicinal or recreational plants and means for real time monitoring, managing and controlling the operation of the system based upon real-time sensed parameters (illustratively temperature, nutrient levels, lighting, mist schedules, CO₂, pH levels and other growth and plant health related items).

Another aspect of the invention is to employ the monitoring and adjustment means to provide data to permit optimization and standardization of the growth and yield of medicinal and recreational plants and non-medical plants to couple to the various grow environment enclosures, to allow remote monitoring and control of each of the grow environment system enclosures, including alerts for each of the real-time sensed parameters.

A still further aspect of the invention is where the telecommunication system comprises a video camera adapted to transmit images from within the grow environment enclosure.

A further aspect of the invention is a climate control system adapted to control the environment within the grow environment enclosure.

A yet further aspect of the invention is a water circulation and storage system adapted to couple to the nutrient delivery system.

Another aspect of the invention is a CO₂ monitoring, controlling and enrichment system.

It is yet another aspect of the invention to provide a curing chamber for harvested flowers to control the temperature and humidity and provide cure controls to permit normalization, standardization and consistency in the efficacy of medical plants for therapies.

A still further aspect of the invention is providing a climate control system for each of the grow environment enclosures and interactive controls between them.

It is yet a further aspect of the invention to provide light of a certain wavelength spectrum produced by light emitting diodes to enhance the photosynthesis of the plants in order to speed up the growth rates and production quantities of plants.

It is yet a further aspect of the invention to distribute the light sources in the grow chamber to provide illumination of the plants from all sides in order to maximize leaf, flower and fruit development and to reduce the variability of the chemical composition of the final product that is typical for conventional grow methods

A further aspect of the invention is to provide for the preparation of some or all of the nutrient solutions according to the needs of the growing crops in a fully automatically controlled system.

It is a further aspect of the to provide for a processor control module includes a processor unit and a storage unit for storing a database of plant growing environment parameters including but not limited to temperature, nutrient levels, lighting, misting schedules, CO₂, pH levels and other growth and plant health related items.

The above and other objects, features and advantages of this invention will be better understood when taken in connection with the following description which is given as exemplary and not limitative.

### BRIEF DESCRIPTION OF THE DRAWINGS

FIG. 1 is a diagrammatic representation of an flow chart of an example of a cultivation and manufacturing system for an integrated plant growth and curing environment system in accordance with an embodiment of the invention.

FIG. 2 is a diagrammatic representation of an oblique front view of an example of an assembled configuration of a tissue incubator plant growth environment system in accordance with an embodiment of the invention.

FIG. 3 is a diagrammatic representation of an oblique front view of an example of an assembled configuration of a nursery plant growth environment system in accordance with an embodiment of the invention.

FIG. 4 is a diagrammatic representation of an example of an assembled configuration of a plant growth for flowering plants in accordance with an embodiment of the invention.

FIG. 5 is a top view of a plant grow environment system for flowering plants in accordance with and embodiment of the invention

FIG. 6 is a rear view of an interior configuration of a plant growth environment system (with the rear panels removed) in accordance with an embodiment of the invention.

FIG. 6A is an example of a rear view of an interior configuration of a plant growth environment system (with the rear panels removed) in accordance with an embodiment of the invention.

FIG. 6B is an exploded view of a cooling assembly for a configuration of a plant growth environment in accordance with an embodiment of the invention.

FIG. 6C is an exploded view of an evaporator assembly for a configuration of a plant growth environment in accordance with an embodiment of the invention.

FIG. 7 is a side view of a plant growth environment system in accordance with an embodiment of the invention.

FIG. 8 is diagrammatic representation of a side view of an example of an assembled configuration of a plumbing system for a plant growth environment system in accordance with an embodiment of the invention.

FIG. 9 is a diagrammatic representation, in exploded form, of an example of a root box assembly of a plant growth environment system in accordance with an embodiment of the invention.

FIG. 10 is a diagrammatic representation, in assembled form, of an example of a nutrient delivery assembly of a plant growth environment system in accordance with an embodiment of the invention.

FIG. 11 is a diagrammatic representation, in exploded form, of an example of a nutrient delivery assembly of a plant growth environment system in accordance with an embodiment of the invention.

FIG. 12A is a diagrammatic representation of an example of a secure remote monitoring nutrient delivery system for a plant growth environment system in accordance with an embodiment of the invention.

FIG. 12B is a diagrammatic representation of an example of a secure remote control nutrient delivery system for a plant growth environment system in accordance with an embodiment of the invention.

FIG. 13 is a block diagram of an example of a control and nutrient delivery system for a plant growth environment system in accordance with an embodiment of the invention.

FIG. 14 is a block diagram of an example of a control and nutrient delivery system for a plant growth environment system in accordance with an embodiment of the invention.

FIG. 15A is a front view of an example of an assembled configuration of a curing environment system in accordance with an embodiment of the invention.

FIG. 15B is a top view of an example of an assembled configuration of a curing environment system in accordance with an embodiment of the invention.

FIG. 15C is a side view of an example of an assembled configuration of a curing environment system in accordance with an embodiment of the invention.

FIG. 16 is a block diagram of an example of a control and monitoring system for a plant growth environment system in accordance with an embodiment of the invention.

FIG. 17 is a block diagram of an example of a control and monitoring system for an integrated plant growth and curing environment system in accordance with an embodiment of the invention.

### DETAILED DESCRIPTION OF AN EMBODIMENT OF THE INVENTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In a preferred embodiment there are a number of major subsystems to the integrated plant growth and curing environment system in accordance with the invention.

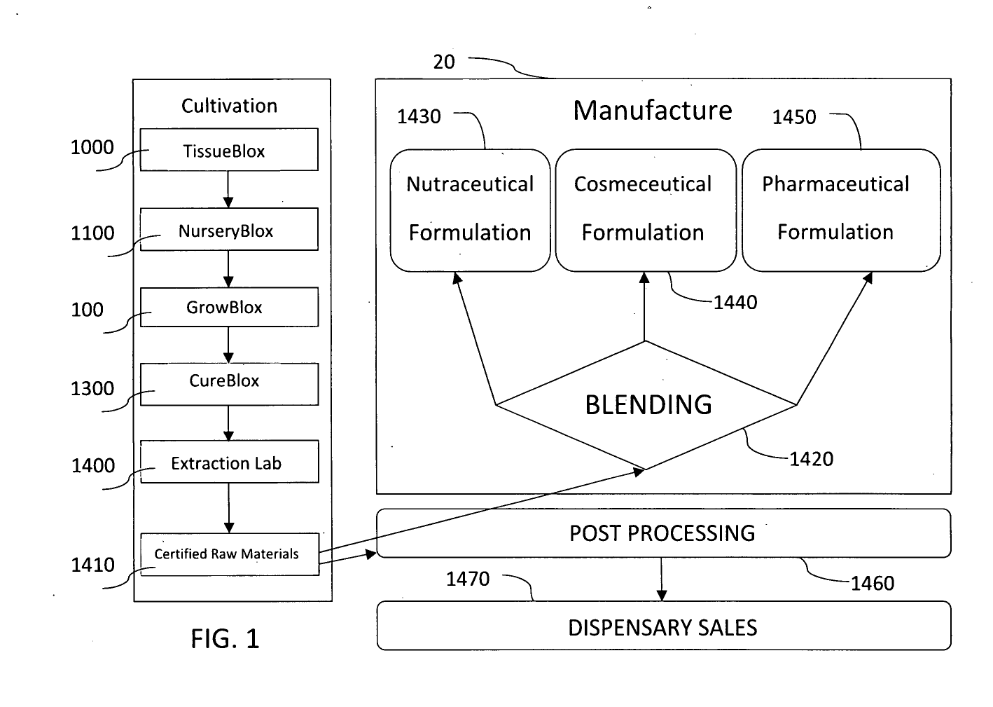

Referring to FIG. 1, there is shown a diagrammatic representation of an flow chart of an example of a cultivation system 10 and manufacturing system 20 for an integrated plant growth and curing environment system in accordance with an embodiment of the invention.

In the particular cultivation system 10, there are a number of operational systems integrated into a cultivation suite. These include a tissue incubator plant growth environment system 1000 (“TissueBLOX”), a nursery plant growth environment system 1100 (“NurseryBLOX”), a vegetating growth environment system 1200 (“VegBLOX”) chamber for plants taken from the NurseryBLOX 1100 and held until they are approximately 24-30 inches in preparation for flowering, a growth chamber environment system 100 (“GrowBLOX”) for flowering plants and a curing incubator 1300 (“CureBLOX”) for curing of harvested flowers.

The cultivation system 10 has, as one of its objectives, to provide cured flowers having a repeatable and optimized potency and efficacy to an extraction laboratory 1400 where the medicinal aspects and ingredients of the plant are removed and subsequently certified based upon pre-established medical and chemical characteristics and requirements. Certified raw materials 1410 are delivered to a manufacturing environment 20 where blending 1420 of the certified raw materials 1410 is undertaken to create one or more formulations from the certified raw materials 1410. Thus, for example, the certified raw materials 1410 may be used to create nutraceutical formulations 1430, cosmeceutical formulations 1440 and/or pharmaceutical formulations 1450.

The certified raw materials 1410 may also be subjected to various post production processing steps 1460 and then the resultant product or products may be provide to purchasers via dispensary sales 1470, either from the cultivator/manufacturer or through any of a number of legitimate dispensary sales 1470 outlets.

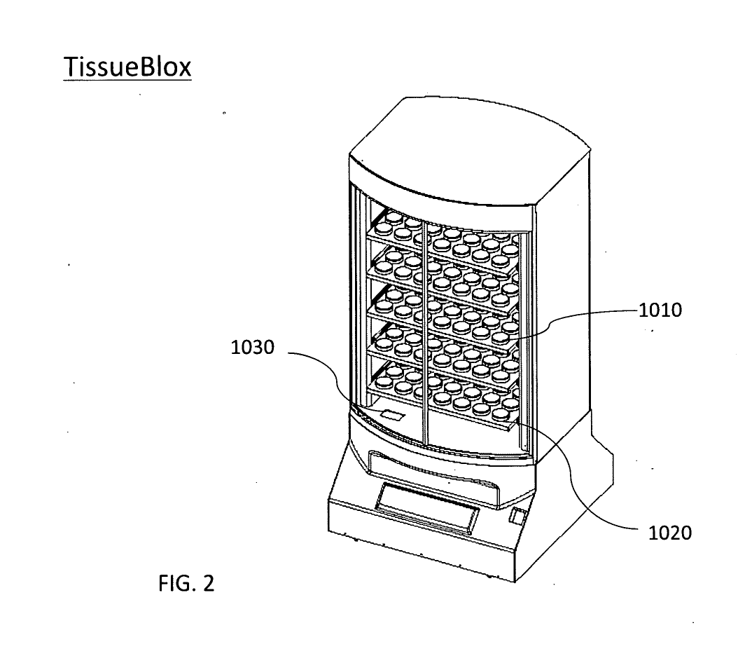

Referring to FIG. 1 and FIG. 2, there is shown an illustrative integrated cultivation system 10 wherein the TissueBLOX 1000 has disposed therein a multiplicity of Petri dishes 1010 and/or magenta boxes (standard plant tissue culture chambers, not shown). Ideally, there is one TissueBLOX 1000 associated with each cultivation system 10 such that the output of the TissueBLOX 1000 is sufficient to provide product for a multiplicity of NurseryBLOX 1100, VegB LOX 1200 and GrowBLOX 100 growth chambers as an integrated unit.

As can be seen and is exemplified by FIG. 2, the TissueBLOX 1000 is an incubator for tissue culture vessels, such as the Petri dishes 1010. The TissueBLOX 1000 has horizontally disposed therein a number of shelf assemblies 1020, each capable of supporting a number of culture vessels. The TissueBLOX 1000 has data acquisition sensors 1030 that monitor environmental conditions, which can be adjusted on-site or remotely. In addition to producing genetically consistent and disease-free plantlets for the grow operation, the TissueBLOXT™ 1000 module continually reproduces and maintains the genetic stock, thereby achieving consistently replicable resultant plants and concomitant medical therapies and patient benefits.

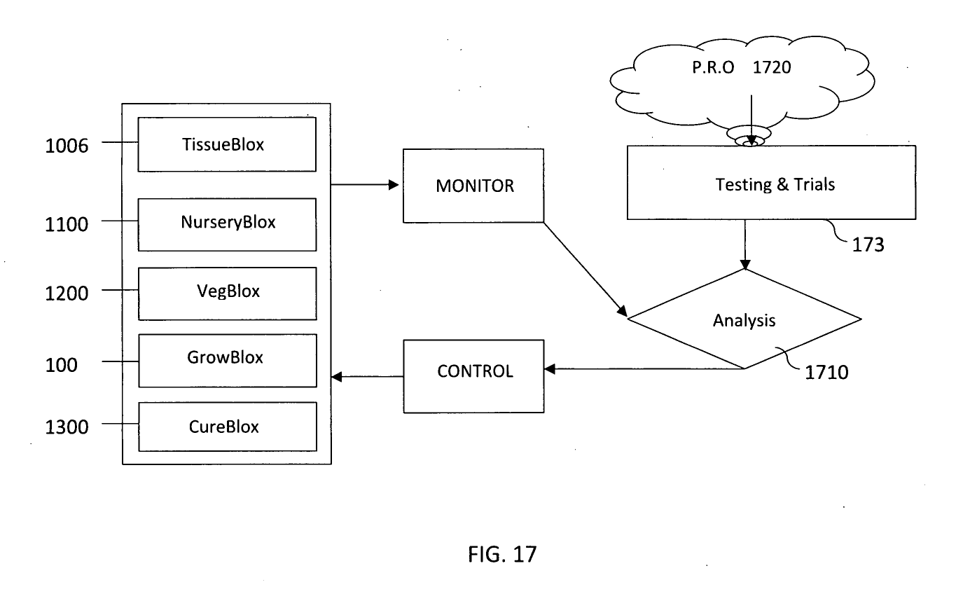

The data acquisition sensors 1030 may to employed to monitor, among other things, temperature, light and humidity. As is illustrated in FIG. 17, the information from the data acquisition sensors 1030 is transmitted to a monitor processor 1710 which records the data, buffers it if necessary and stores it in a data storage or processor or other data handling means. The data (along with other data from the other components of the system), may then be employed to perform analytics which, in conjunction with testing and trials, may be used to correlate the profiles of active ingredients of a medical use plant with symptom and disease-specific improvements. By way of example only, in order to treat specific disease categories with Cannabis-based products, the active ingredients, which include both cannabinoids and terpenoids, are monitored and their concentrations evaluated and controlled through changes to the plant growth conditions.

The analytics permit, for each individual Cannabis strain, a measurement of the “profile” of active ingredients in the laboratory. The analytical techniques define the amounts and kinds of active molecules in that strain, which have been shown to be effective in the treatment of conditions including pain management, mood, metabolism, inflammation, cancer, neural and immune disorders. The cannabinoid and terpenoid profiles of a representative strain are depicted here.

The TissueBLOX 1000 is employed in connection with the system where tissue propagation is desired. The tissue propagation is the process of mass producing genetically identical progeny plants from a single tissue sample taken from a carefully selected parent plant or cultivar in a sterile environment. The TissueBLOX 1000 permits the tissue propagation to retain the advantages of identity of progeny and the ability to propagate identical medical use mini-clone plants, while having the additional advantage that only a small piece of tissue from the cultivar or parent plant is required and additionally permits the rapid production of plant cells to accelerate the time that the plant becomes flower bearing and therefore generates the useful component for medical purposes.

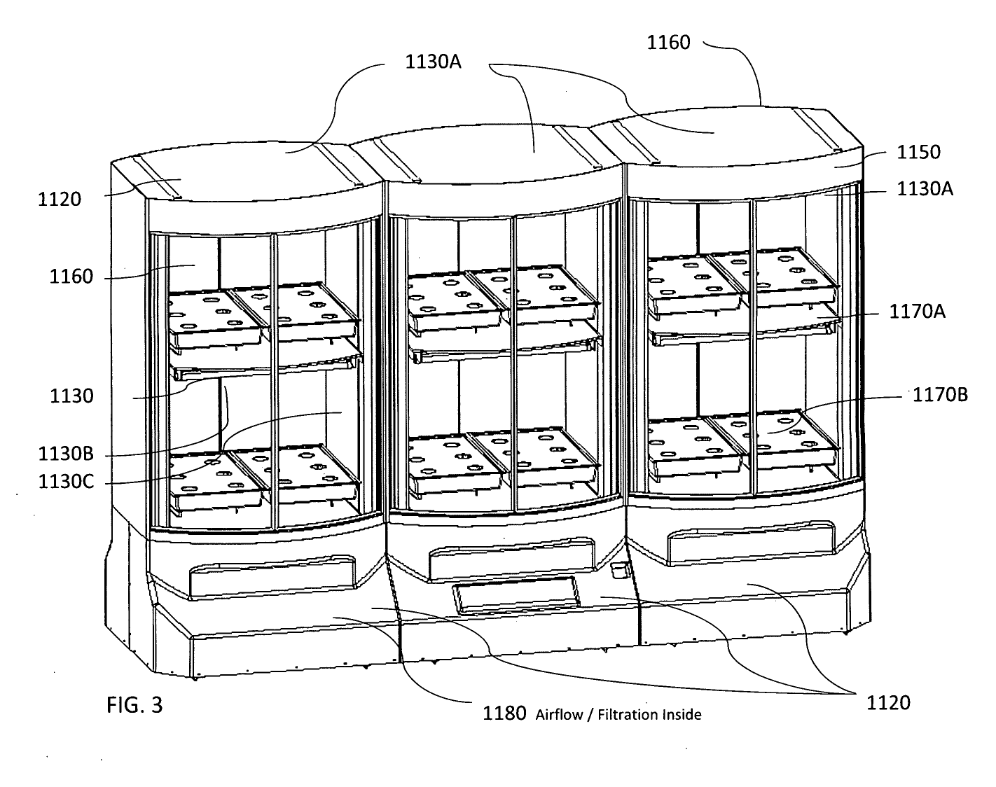

Referring to FIG. 1 in conjunction with FIG. 3 and FIG. 17, there is shown an illustrative integrated cultivation system 10 wherein the NurseryBLOX 1100 has disposed therein a series of plant rooting trays 1110 for rooting mini-clones from tissue cultures that have been generated and controlled within the TissueBLOX 1000. Alternatively, the NurseryBLOX 1100 may also be advantageously used to start cutting from one or more mother plants.

It will be further appreciated that the description of the NurseryBLOX 1100 and the VegBLOX 1200 have certain common aspects and environmental controls that permit the NurseryBLOX 1100 to be employed for certain vegetative growth phases during the time that it is being employed to accommodate the mini-clones referred to above. Accordingly, the VegBLOX 1200 and the NurseryBLOX 1100 will be hereinafter described together and referred to as the Nursery/Veg Center. FIG. 3 is illustrative of the Nursery/Veg Center for purposes of the following description. It will be appreciated that the two may be employed separately, or a single unit may serve both functions or a single unit may serve each function successively based upon the needs of the user, the size of the plants and the stage of the growth cycle.

The Nursery/Veg Center is a fully contained unit for environmental control that may be advantageous employed. It is designed to produce starter plants and to accommodate plants during the vegetative growth phase, before transfer to the GrowBLOX 100 for blooming. It consists of three concurrently controlled grow compartments 1170. Each compartment 1170 has an upper level 1170A for starter plants and a lower level 1170B for vegetative grow.

There may be common controls for both levels which permit the temperature to be maintained at 20-30° C. (68-86° F.) and the humidity range at between 20%-80%. In addition, a CO2 supply is generally needed to accelerate growth and may also be employed to provide an insect-killing option. The CO2 supply may be operated at a range of from 400 ppm (ambient) to 7,000 ppm (temporary max for insect control).

Additionally, there is air flow/filtration system 1180 substantially similar to that which will be discussed in relation to the GrowBLOX assembly and system to provide air circulation for even distribution of temperature, CO2 and humidity throughout the Nursery/Veg Center. It is a further feature of the Nursery/Veg Center that carbon filtration may be employed to control odor. It is yet a further aspect of the invention that advanced filtration may be employed for capturing terpenoids prior to the curing and processing of the flowers.

Referring again to FIG. 3, there is shown the upper level 1170A which is also referred to overall as the nursery section 1190. The nursery section 1190 may have rooting compartments 1192 situated on each of the upper levels 1170A. The Nursery/Veg Center may have several modes of operation and employed for various function. It may be used for rooting of tissue-culture micro-shoots for transfer to vegetative growth (lower level) compartment 1195. It may also be use for rooting of shipped micro-clones for conventional or GrowBlox cultivation. Alternatively, it can also be employed in the production of micro-clones for shipment (plantlets that have no roots yet but will in a few days) and, in the event that there is excess capacity at any point in time, it can be used for growing small, auto flowering cultivars.

The Nursery/Veg Center is where the micro-clones from the TissueBLOX 1000 are transferred from a sterile to non-sterile but sanitary environment. In one preferred embodiment, the plantlets (not shown) are retained in Rockwool plugs or cubes of approximately 1″ diameter/width and remain in the rooting compartments 1192 of the nursery section 1190 for approximately 1-4 weeks.

The lighting arrays 1130A in the upper level 1170A may be comprised of LED lights mounted above the plants at a distance of between 12-18″. It is preferable to employ full spectrum LED lights that have an adjustable light intensity at 6″ below the light array 1130 of 50-300 μmol×m·²×s¹PAR (photosynthetically active radiation).

In a preferred embodiment of the invention, the plantlets are in baskets that may be filled with clay pellets or other similar material and are placed in the rooting compartments 1192. The depth of each of the rooting trays 1110 within the nursery section 1190 is approximately 4″ and each tray 1110 is adapted to hold standard baskets for transfer to lower level 1170 B (which is the vegetative growth (lower level) compartment 1195) and subsequently the GrowBLOX 100.

The humidity, watering and nutrients in the Nursery/Veg Center are controlled and monitored in order to ensure that the plantlets are adapted to LED light and aeroponic watering. The Nursery/Veg Center employs spray-nozzle watering and has a programmable feed cycle and an ability to provide mist to the plantlet rooting compartments 1192 in droplet of 30-100 μm. The upper level 1170A has associated therewith an upper level main tank (not shown) that contains the nutrient feed from peristaltic pumps and the supplement feed water with rooting-inducer. The upper level main tank may also be employed to provide pH adjustment, control the water temperature (which optimally should be at 20-25° C. (68-77° F.)) and has sensors to permit environmental control data to be recorded.

The upper level 1170A also has associated therewith an upper level drain tank (not shown) for collecting the excess feed water and recycling from drain tank to the feed pumps. It is also feasible to provide a drain-to-waste option with the drain tank.

The Nursery/Veg Center has a variable capacity depending on the density of the plants, the height of each plant and the species of plant with its particular requirements. As is illustrated in FIG. 3, the Nursery/Veg Center may be comprised of from 2 to 4 levels and hold up to 24 plantlets per level. The upper level 1170A will generally have a larger number of plantlet associated therewith while the lower level 1170B will generally have a smaller number of plantlets in each chamber and on each level. By way of illustration, the lower level 1170B may advantageous contain 4 root boxes/compartment=12 total/box, or 1 root box with adapter tray that can hold 4 plants with its own spray assembly.

The lower level 1170B is also referred to as the vegetative growth chamber or level. Depending on the plant, the optimization factors, the growth rate and other variables, the vegetative growth level may be an entire Nursery/Veg Center or may be only one or more shelves within such a center. The lower level 1170B is ideally for growing plants at long light cycles in preparation for transfer to a GrowBLOX 100. Within the lower level 1170B, the watering may be a combination of spray (30-60 μm droplet size mist) and fog (5 um droplet size). The upper level 1170B has a separate feed timing and nutrient composition from the upper level 1170A.

The lighting arrays 1130B in the lower level 1170b may be comprised of LED lights mounted above the plants at a minimum distance of 24″. It is preferable to employ full spectrum LED lights that have an adjustable light intensity at 6″ below the light array 1130 of 50-1,000 μmol×m·²×s¹PAR. It is also advantageous, depending on the plant, to employ full spectrum LED side lights 1130C for the lower level only.

Plants stay in Veg Level for approximately one half the time they stay in the GrowBLOX 100.

In a preferred embodiment there are a number of major subsystems to the self-contained plant growth environment system in accordance with the invention.

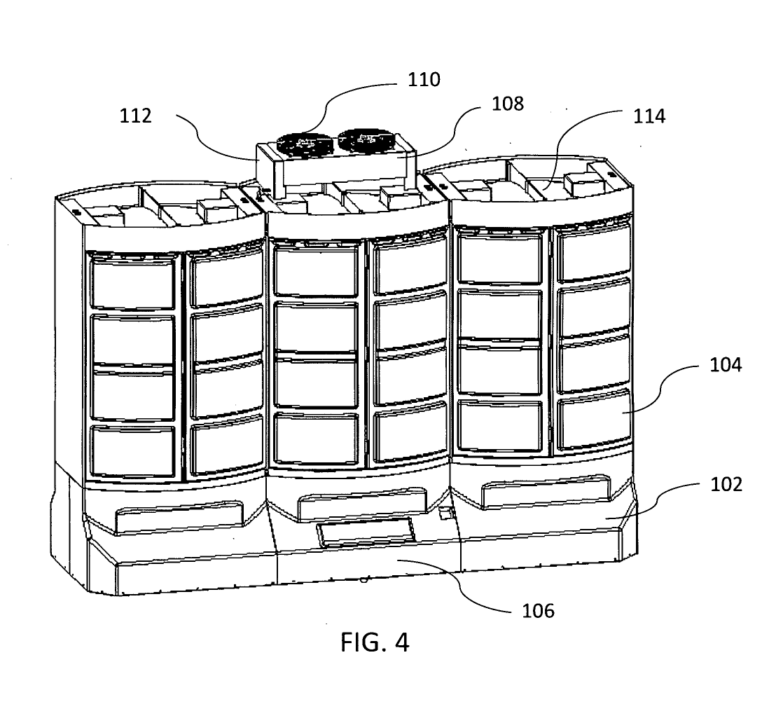

Referring to FIG. 4, there is shown a diagrammatic representation of an example of an unassembled configuration of the GrowBLOX plant growth system 100 in accordance with one embodiment of the invention. In the particular delivery system 100, a number of the operational elements of the system such as cooling system 200 and evaporation system 300 are is covered by protective covers 102 in order to maintain the self-contained aspect of plant growth system 100.

Additional protective door covers 104 are advantageously provided to further enclose the plant growth system 100 in the area where the plants (not shown) are generally maintained and to further serve, on the interior surfaces thereof, as reflected internal elements for a system of light emitting diodes 400 in accordance with and embodiment of the invention.

Referring to FIGS. 4-7, a nutrient coverlid 106 is removably deployed above the nutrient dispensing trays (not shown) that are provided within the plant growth system 100. Air conditioning condensing unit 108 is illustratively deployed above the plant growth system 100 and has disposed therein one or more condenser fans 110 and has associated there with one or more return here ducts 112 to provide the air delivery and return system for the plant growth system 100. Upper ceiling covers 114 are dispose over each of the individual units of the plant growth system 100 in order to provide a fully enclosed environment for the plants that are to be grown within the plant growth system 100.

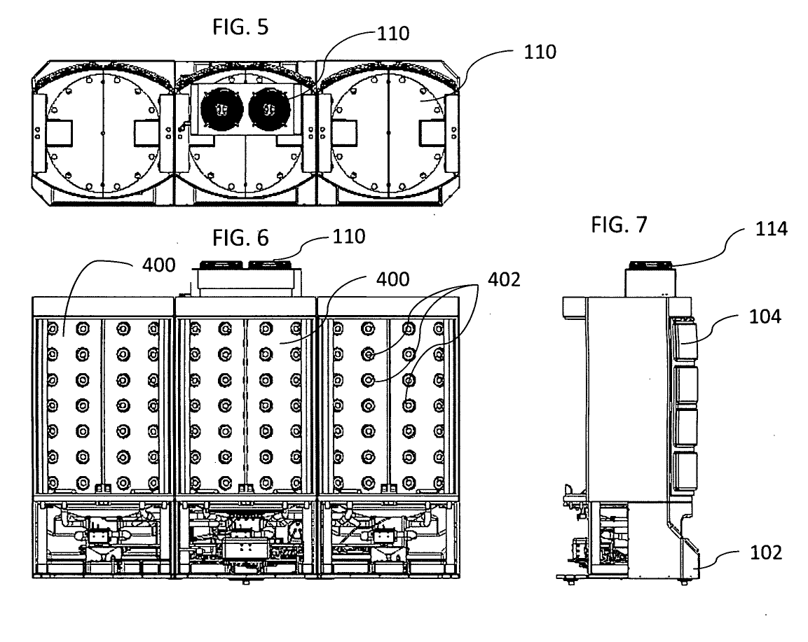

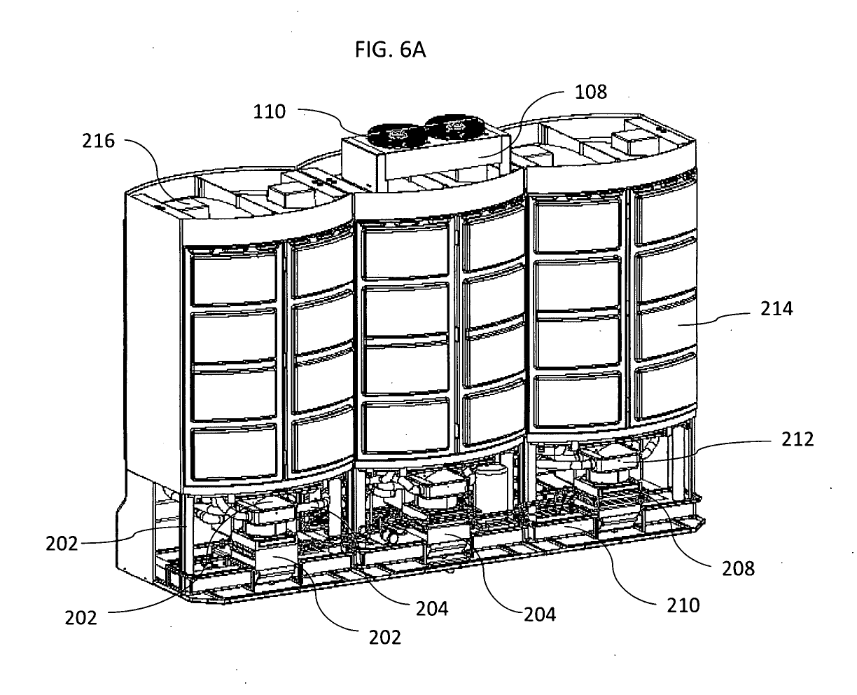

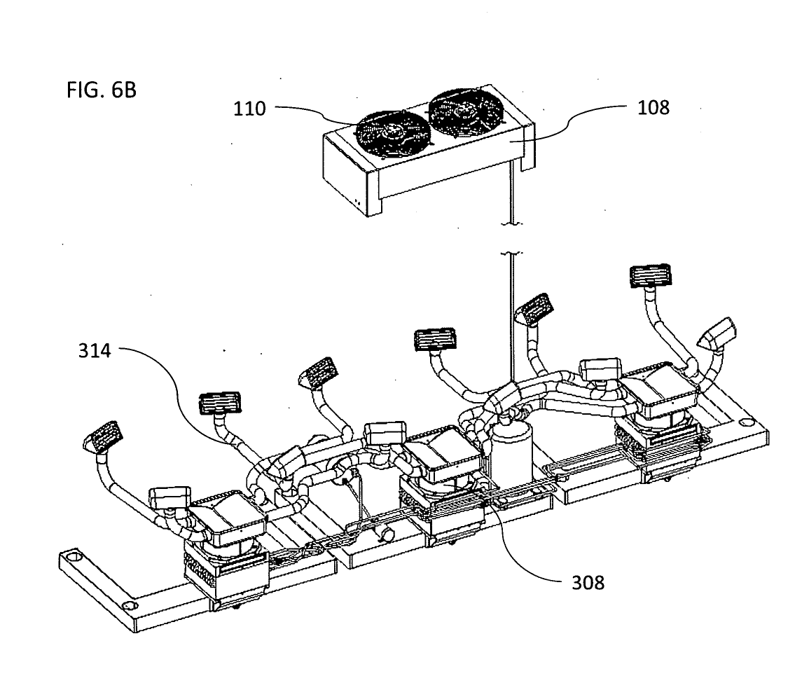

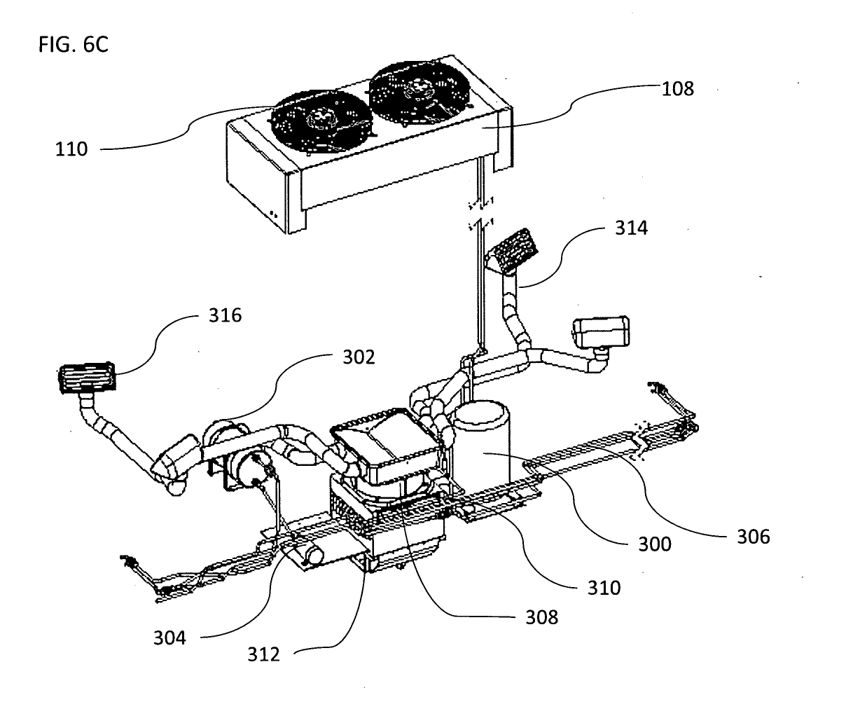

Referring to FIG. 6, there is illustratively shown the plant growth system 100 with the protective doors 104 removed in order to show the system of light emitting diodes 400 advantageously deployed within each of the units of the plant growth system 100. As will be further explained hereinafter, the system of light emitting diodes 400 consisting of a plurality of light emitting diodes 402, may be provided with variable wavelength diodes 402 in order to permit the plants to receive optimum light in accordance with the requirements of the particular plant which is being grown within the plant growth system 100.

Referring to FIG. 6 in conjunction with FIGS. 6A, 6B and 6C as well as FIG. 7, there is shown an illustrative embodiment of the plant growth system 100 in conjunction with its related cooling system 200 and evaporation system 300. As is best illustrated in FIG. 6A, and evaporate or glycol cooler 202 is employed beneath the plant growth system. A variable speed fan 204 provides the airflow through the plant growth system 100. The variable speed fan 204 is capable of providing multiple air flows through air supply tubes 206 which are advantageously deployed within each of the units of the plant growth system 100.

The cooling system 200 air supply which is furnished to the plants is further enhanced by filtering the air through a HEPA filter (not shown) advantageously situated at HEPA filter port 208. The cooling system 200 air supply is provided into each of the one or more units comprising the plant growth system 100 and is returned via bottom air return ducts 210. The cooling system 200 is further provided with an air supply register 212 which maybe deployed in connection with each of the one or more units comprising the plant growth system 100.

The plant growth system 100 is advantageously provided with rear mounted doors 214 each of which has disposed thereon a plurality of light emitting diodes 402. It will be appreciated that the rear mounted doors 214 maybe removably disposed in order to permit access to the plants and, the front mounted protective doors 104 make similarly be provided with a plurality of light emitting diodes 402 in order to provide a full surround of lights to the plants within the growth delivery system 100. Referring still to FIG. 6A there is also shown a series of carbon filter housings 216 disposed at the upper level of each of the units within the plant growth system 100 to provide additional particulate matter and ambient odor removal.

Referring again to FIG. 6B, the air-conditioning condensing unit 108 having condenser fans 110 is connected to an air-conditioning compressor unit 218 which serves to provide constant temperature cool air at the temperature determined by the operator to be optimal for the particular plant and the particular phase of growth for that plant within the plant growth system 100. As will be discussed at a later point in this specification, each of these units and others associated with the plant growth system 100 may be controlled both proximately and remotely by the operator and are further controlled through sensors that are advantageously employed to determine such variables as carbon dioxide level, nutrient flow, humidity, and other applicable parameters to ensure maximum growth and viability of the plan at each stage of its growth cycle.

Referring to FIG. 6C, the evaporator system 300 is comprised of that evaporator glycol cooler 302 that is functionally connected to a glycol pump 304 which distributes the glycol through a series of freon line 306 through each of the units of the plant growth system 100. A variable speed fan 308 is juxtaposed above a HEPA filter port 310 which sits above the bottom air return duct 312. In operation, the air is circulated through one or more air supply tubes 314 and out adjustable directional air vents 316 within each of the units that form a part of the plant growth system 100.

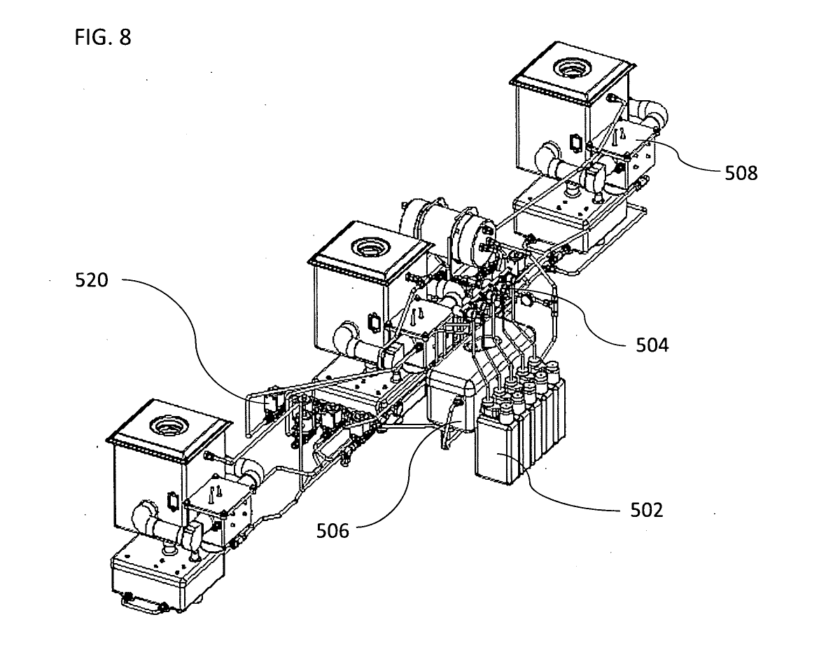

Referring to FIG. 8, there is shown exemplary plumbing structure 500 for providing the nutrients, mist and other deliverables to the plants as well as obtaining data from plants and environment in order to provide control functions. A series of nutrient bottles 502 are disposed in connection with the plumbing structure 500, each of which provides one or more designated nutrients. Each of the nutrient bottles 502 maybe individually regulated or regulated in connection with other nutrient bottles 502 in order to supply optimal nutrients to the plants within the plant growth system 100. Each of the nutrient bottles 502 is connected to a peristaltic pump 504. A main water tank reservoir 506 is integrally connected to and AeroVapor nutrient and H2O delivery unit 508 which mixes and delivers the combination of water and nutrients to the plants within the plant growth system 100.

As will be further explained hereinafter, the AeroVapor nutrient and H2O delivery unit 508 is controlled through the further part of the invention via a series of control and feedback loops and related optimization sensors that create an ongoing and continuously updated set of parameters in order to provide the optimal nutrient and water combination to the plants during each phase of their growth cycle.

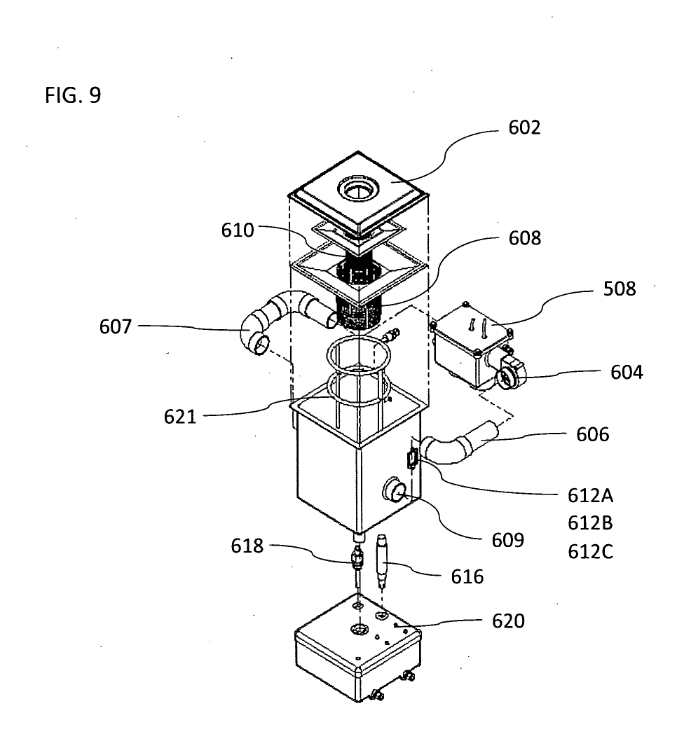

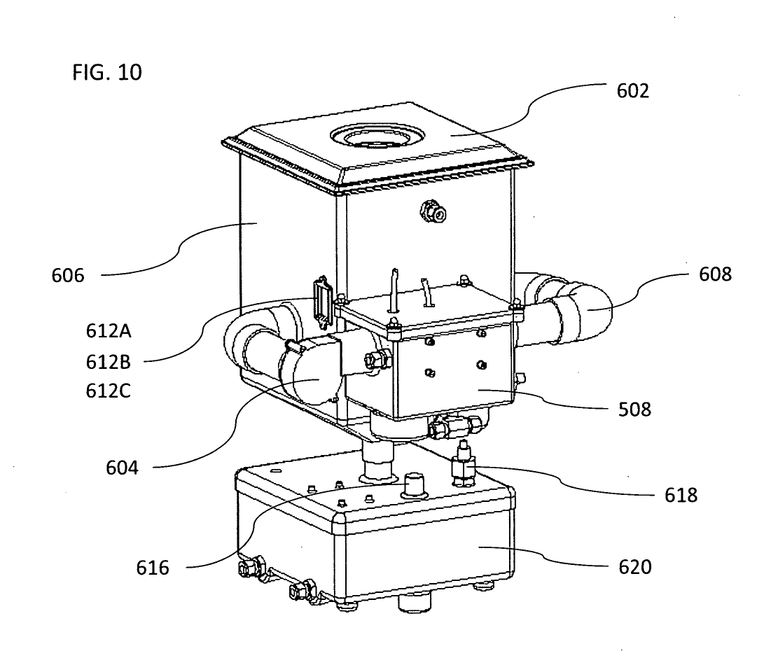

Referring to FIG. 8 in conjunction with FIG. 9 and FIG. 10, there is shown an exploded view of a root box assembly 600 which has as a part thereof the AeroVapor nutrient and H2O delivery unit 508. The root structure of a plant (not shown) is placed within the root box assembly 600 such that the roots are generally contained by the root box assembly 600 and are below the level of an airtight root box chamber cover 602. The AeroVapor nutrient and H2O delivery unit 508 is connected through a blower fan assembly 604 to an inflow tube 606 that is integrally connected to the lower portion of the root box assembly 600, below the level of the root box chamber cover 602.

Root baskets are deployed within the root box assembly below the level of the root box chamber cover 602. By way of example, there is shown a large root basket 608 and a small root basket 610 deployed within the root box assembly 600 below the level of the root box chamber cover 602. The inflow tube 606 opening 607 into the root box assembly 600 is advantageously disposed so that the nutrients and mist contact the roots of the plant substantially immediately upon entry into the root box assembly 600 and are disbursed throughout the root structure both by being blown in through the inflow tube 606 and being drawn through by means of an outflow tube 607 that is dispose on the opposite side from the inflow tube 606 at opening 609 and is functionally connected thereto, thus creating a controlled air flow current through the root structure.

A series of sensors are deployed in connection with the root box assembly 600 and may be disposed along its various side and bottom. Advantageously, oxygen 610, humidity 612 and air temperature 614 sensors are shown on a lateral wall of the root box assembly 600, while a pH sensor 616 and an environment control sensor 618 are connected a drain tank 620 that is disposed below the root box assembly 600 and into which falls the unabsorbed water and nutrients. The various above named sensors are only illustrative of the variety of sensors that may be deployed in connection with the root box assembly 600 and with the scope and breath of the invention. It has been found that deploying the sensors in the manner set forth above provides advantages in controlling the overall environment and provided truer data for such control than if the sensors are deployed elsewhere in the root box assembly 600.

The root box assembly 600 is also provided with a double watering ring assembly 621 that may provide water at various levels of the roots that are contained within the large root basket 608 or the small root basket 610. By providing both water and misting, as will be explained next, the roots receive the optimum water and nutrient mix which can be altered on a just-in-time basis predicated upon the information and data provided by each of the sensors and the underlying growth modeling that has been recorded and determined from prior growth cycles for the same species of plant or for other species with similar growth patterns.

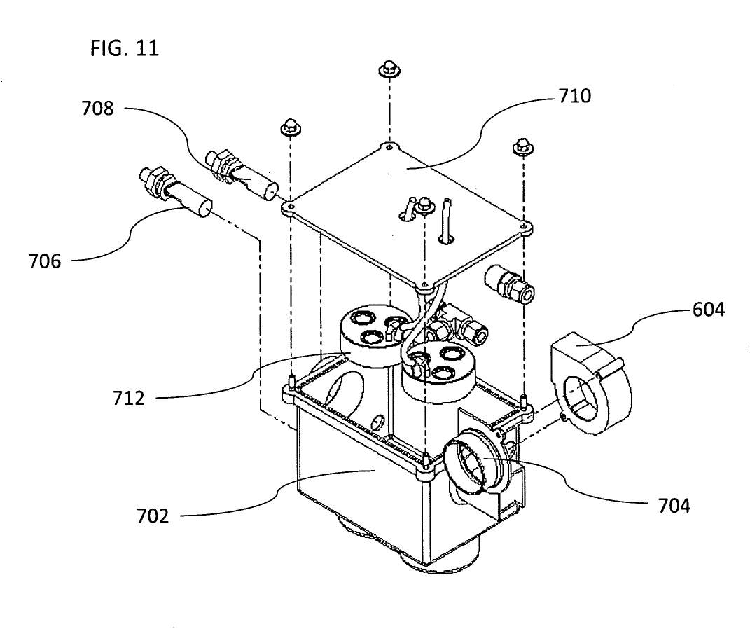

Referring to FIG. 11, there is shown an exploded view of the AeroVapor nutrient and H2O delivery unit 508. The operational elements of the AeroVapor nutrient and H2O delivery unit 508 are housed within a water containment unit 702 into which water and nutrients are placed in a controlled fashion through the operation of one or more of a series of water control solenoids 520 that operate in connection with the plumbing structure 500. The blower fan assembly 604 is shown in exploded form and in proximity to blower fan pipe attachment port 704 that mates to the inflow tube 606 that is integrally connected to the lower portion of the root box assembly 600, below the level of the root box chamber cover 602.

The water level in the containment unit 702 is monitored by an upper level water sensor 706 and a lower level water sensor 708 that cause the water level to be maintained within certain boundaries and ensure constant hydration of the roots in an optimal manner. The containment unit 702 has a water tight top 710 such that the water is capable of being fully controlled by elimination of evaporation, thus permitting the unit to provide substantially exact information as to water uptake as a function of the water sent into the system.

One or more ultrasonic piezo misting units 712 are deployed within each containment unit 702 to create the mist that is picked up in the airflow of the blower fan assembly 604 and distributed to the roots of the plant within the AeroVapor nutrient and H2O delivery unit 508.

In general, the plant growth system 100 may be characterized as a multi-unit grow chamber for flowering plants in which there are controls for temperature, light, humidity, watering, nutrients, CO2, O2 and the capacity for misting roots for eliciting plant stress responses and to deliver peroxide for root health. The plant growth system 100 may also have the capacity for fogging of the roots in such circumstances as may be desirable or needed for controlled stressing of the plants. It has been found that, among other plant species, the plant growth system 100 is advantageously used for the enhanced growth of medicinal plants including cannabis and that the system may be advantageously employed to provide the capacity to filter out and recover terpenes.

Referring once again to FIG. 4 and FIG. 6, there is shown an illustrative surround of LED lights in each of the units of the plant growth system 100 for flower development at all levels. The interior walls of the various protective wall covers 104 and 106 may have disposed thereon the light emitting diodes 400. They may also be reflective either by coating of the interior walls or by using a white reflective material that minimally absorbs the light emitted by the diodes 400.

The chlorophylls of the plants deployed within the plant growth system 100 mainly absorb blue light with a relatively shorter wavelength and red light with a relatively longer wavelength. However, light of other wavelengths is also captured by supporting plant pigments and also contributes to photosynthesis. In order to enhance the illumination efficiency of light absorbed by the plants, the light emitted by the light emitting diodes 400 is covering a spectrum that has been shown to be optimal for photosynthetic activity:

The plants can obtain stable and adequate illumination from the light emitting diodes 400. These produce a full-spectrum light that is optimized for photosynthesis. By adding, exchanging or dimming specific diodes the light spectrum can also be adjusted to increase the production of flowers, fruit, essential oils or other desirable products. Thus the crop can also be increased. Furthermore, because of the light emitting diodes 400 with different optical wavelengths on the interior walls of the chamber are capable of being staged to provide different aggregate light during different parts of the growing cycle, users can deliberately facilitate the growth rate of either leaves, flowers or fruit of the plants in order to increase the crop.

Continuing to refer to FIG. 4 and FIG. 6, it is a further aspect of a preferred embodiment of the invention to employ programmed illumination cycles that use phytochrome modulation to induce flowering of Cannabis plants at light periods of 1 more than 12 hours per/day. By using such phytochrome modulation, the Cannabis plant is capable of growing more rapidly and producing a harvestable crop more quickly, and thereby reducing the length of the cultivation cycle. Phytochrome is a photoreceptor that changes between active and inactive forms in the darkness or in response to exposure to far red-wavelength light and in particular narrow-band, far-red LED lights. The benefit is that plants could be grown at longer daylight hours and still bloom. The configuration of the light-emitting diodes on both around and above the plant increases illumination of the lower parts of the plant that otherwise would be shaded by the upper leaves. This enhances overall flower production, as well as fruit development and ripening.

In another aspect of a preferred embodiment of the invention, oxygen from the atmospheric environment or from a supply may be transferred into the root box assembly 600, which increases root health and nutrient uptake. Oxygen levels in the root box are monitored and adjusted to by supplementing air or pure oxygen from a pressurized source.

Carbon dioxide is absorbed from the air and converted to sugars during photosynthesis. Supplying the growing plants with supplemental carbon dioxide increases the rates of photosynthesis and growth. The enclosed environment allows for adjusting the carbon dioxide concentration in the shoot compartment effectively because only a relatively small volume has to be delivered. Furthermore, temporarily increasing the carbon dioxide concentration can be used as a non-chemical pest control measure.

As can be illustratively seen in FIG. 8. a preferred embodiment of the invention provides separate reservoirs for the individual nutrient solutions and solutions to adjust the pH up or down, all in accordance with the information received from the various monitors employed within the system. Thus, based upon oxygen level, temperature, humidity, carbon dioxide level, pH and other variables the necessary reservoirs are tapped to provide the nutrient solution and the appropriate pH for the plant at each phase of its growth cycle. Each stock solution is prepared in the water tank and transferred to the plants in the form of a nutrient mist that is directly applied to the roots by means of the AeroVapor nutrient and H2O delivery unit 508.

The temperature of the rhizosphere (roots) plays a very important role in plant growth because it is associated with the radical metabolism and assimilation of nutrients. In evolution, various plant species have adapted to different environments, cold or hot in respect of temperature. Consequently, the optimal growth temperature of the rhizosphere differs greatly among plant species, and even between cultivars of the same plant species. The regulation and control therefore of the rhizosphere temperature for the growing and harvesting of a crop is an important and critical aspect of the present invention.

A major advantage of the present invention is the automatic regulation and control of the temperature in the root zone, which is achieved by adjusting the temperature of the nutrient solution that is administered to the roots. Thus, the optimal temperature for each phase of the grow cycle can be maintained regardless of the temperature outside the device. The system has the ability to regulate the temperature of the supplied nutrient solution separately for the plant being grown and the crop which it is expected to bear.

Similarly, the temperature and humidity surrounding the crop bearing portion of the plant is important to the optimal growth and crop production. If humidity is too high, the crop may rot, while if it is too low it may dry out or not reach maximum development. Once a crop is stunted because of inclement surroundings, it may often never recover or reach its optimum potential. As will be set forth hereinafter, it is yet another aspect of a preferred embodiment of the invention to provide constant monitoring and adjustment of the multiple variables that will enhance and optimize a plant's productivity while also providing data to determine the best conditions for future maximum yield. It is a part of the invention to provide a learning model system for control of the plant growth system 100 that teaches itself based upon past data derived from within the plant growth system 100, current crop data as sensed by the plurality of sensors and crop data derived from outside of the plant growth system 100 including environmental and natural growth data.

The present invention provides for an integrated incubation, cultivation and vegetation system, with automatic root irrigation system providing the nutrient solution by pumps, transport pipes and misting under pressure (high or low) directly to the root inside root containers in those portions of the system where the plants have roots or are tending to grow roots. The system is advantageously provided with automatic setting of time and frequency of mist provision based upon stored data and currently sensed data. The nutrient solutions for the plant growth system 100 are both a closed circuit supply system, recirculating the nutrient solution that is not absorbed by the plants from the growing baskets back to the drain tanks where the resultant concentrations and nutrient values may be determined, as well as an open circuit system to replenish and correct nutrient values prior to delivery of the nutrients to the roots.

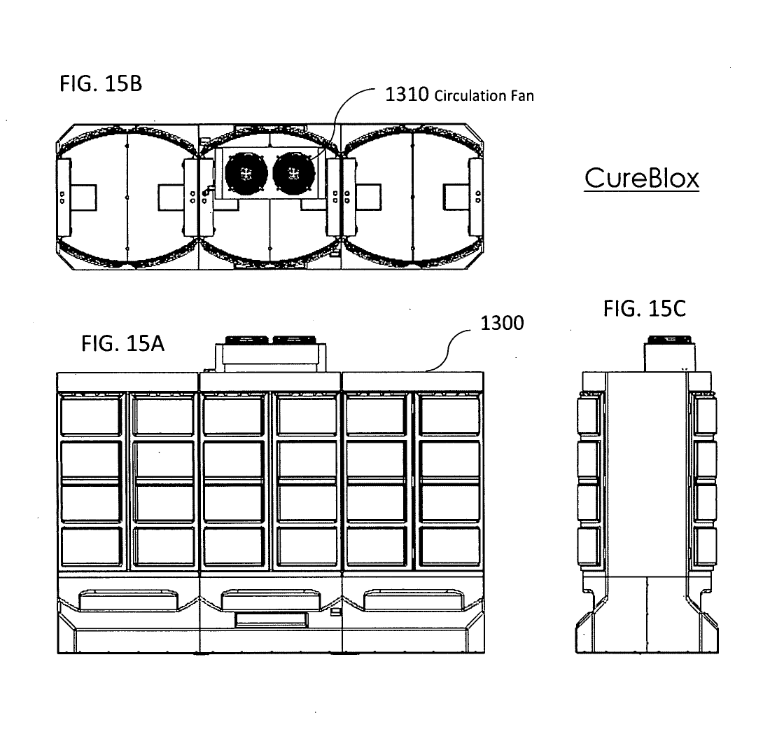

Referring to FIGS. 15 A and 15 B, there is shown an example of a CureBLOX 1300 curing system that provides the environmental conditions necessary for removing moisture from harvested plant material, such as flowers, leaves, fruit or seeds and simultaneously inactivates fungal spores and thus prevents mold growth. The CureBLOX™ 1300 cabinets monitor adjust the ambient humidity, temperature, and air flow inside the chamber according to a programmed sequence. As an example, the curing process preserves the medicinal cannabinoids and terpenes of harvested Cannabis flowers while inactivating any mold spores or other microbial contaminants. The curing process usually takes 1-2 weeks and involves changing the temperature and humidity in the chamber simultaneously at fast or more gradual rates. Critical for the process is that these changes occur reproducibly and evenly throughout the entire volume of the chamber and can be programmed and customized according to the type and initial condition of the plant material to be cured.

As can best be seen by referring to FIGS. 15 A and 15 B, there is shown a diagrammatic representation of an example of a configuration of the CureBLOX 1300 in accordance with one embodiment of the invention. In the particular system 1300, a number of the operational elements of the system such as cooling system, heating system and evaporation system are covered by protective covers 1310 in order to maintain the self-contained aspect of curing assembly 1300. The CureBLOX 1300 has circulation ducting (not shown) through which both cooling and heating airflows may be advantageously deployed. The CureBLOX 1300 is connected to a control and monitoring system exemplified in FIG. 17 through an HMI panel human interface. The HMI permits the creation of alternative curing cycles based upon the nature of the plant material being dried, its medical characteristics and products, the humidity (moisture/water) in the material and the ultimate product to be obtained from it.

It a preferred embodiment, the CureBLOX 1300 also maintains a mold eradication curve for up to 40 pounds of wet product and has the capacity to remove up to 15 pounds per day of water, depending on product load and a 4 pound per hour dehumidification capacity. It also has a cooling capacity which, in one embodiment, is approximately 1,500 btu/hours while also maintaining a heating capacity of up to 4 KW. The above heating and cooling capacities are merely illustrative of a CureBLOX 1300 chamber and may be either higher or lower depending on the chamber size and configuration, the type and condition of plant material to be cured the resultant end product.

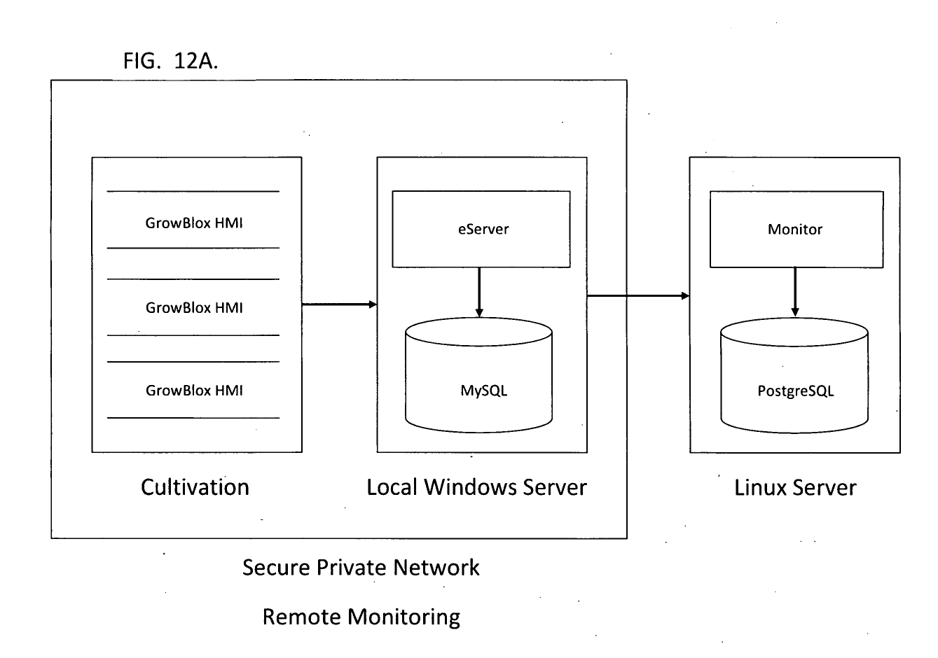

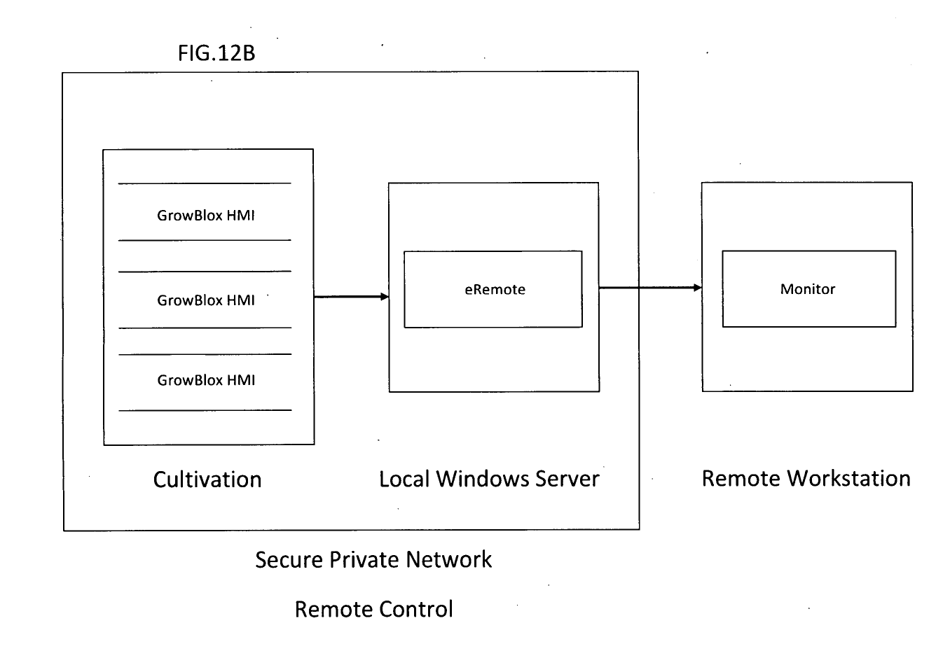

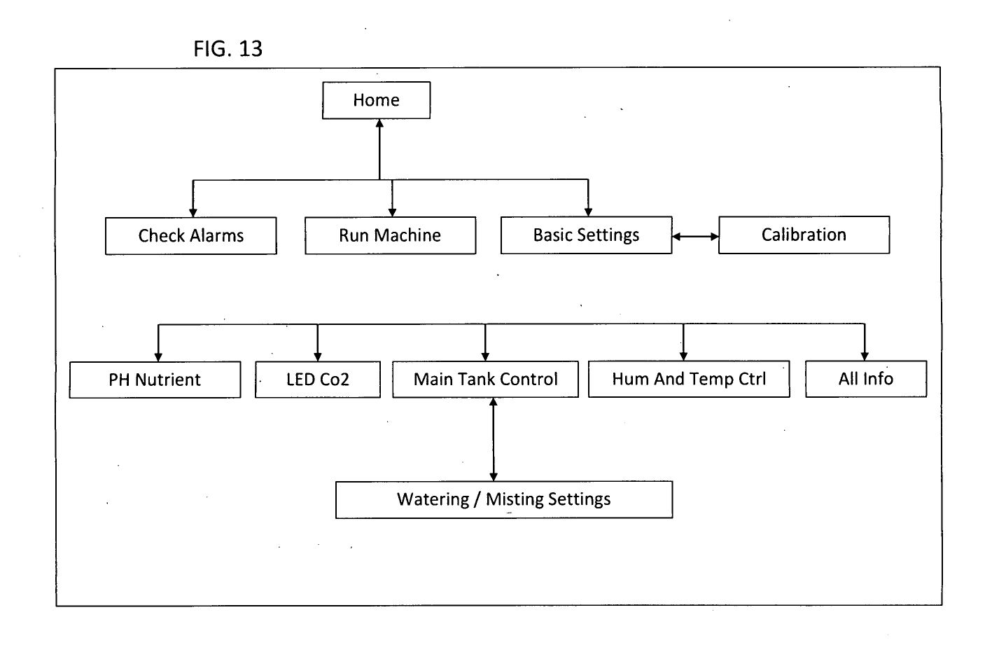

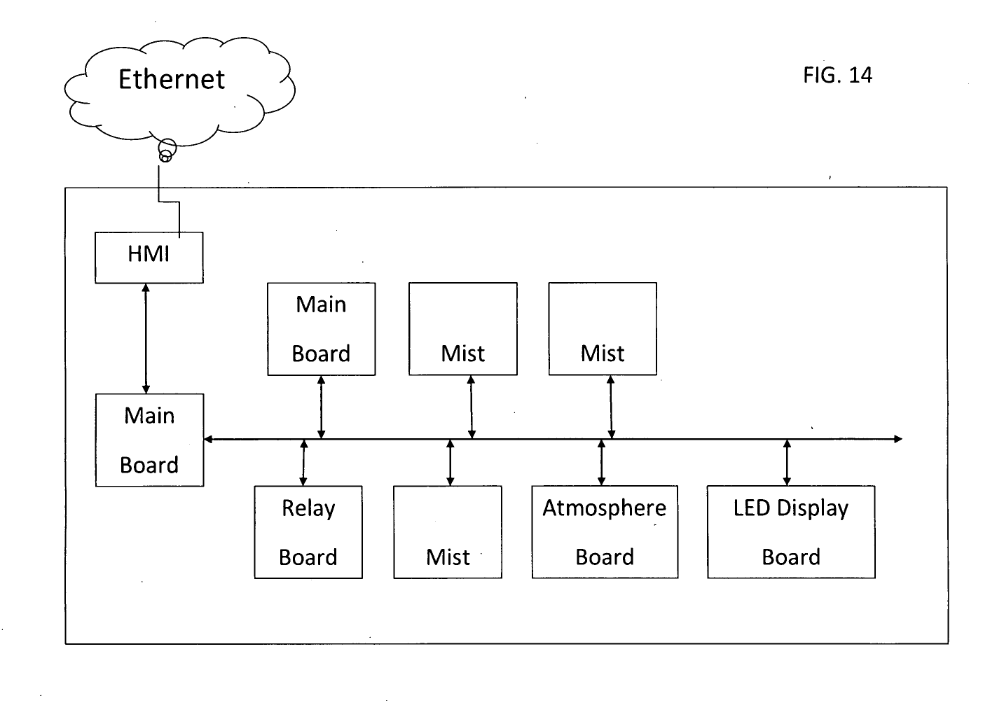

Referring to FIGS. 12A and 12B, in conjunction with FIGS. 13 and 14, there is illustratively shown a flow chart for a central automatic digital control system that may be operated by computer to provide monitoring and control of all of the individual parts of the system, and permit remote, on-line control.

The functional elements of FIG. 14 are:

| PCB board | number | Function |
|---|---|---|
| Main board | 1 | Communicate with slave boards, and control all |
| | | the components to set the GrowBlox run as |
| | | schedule. |
| Relay board | 1 | With 32 relays control most of the components in |
| | | the system |
| Fog Tank | 3 | Get root box hum/temp/O₂ data |
| | | Get fog tank water level |
| | | Get drain tank water level |
| | | Control piezo mister, mister fan |
| | | Control O₂ Solenoid |
| Main tank | 1 | Get water level of main tank |
| | | Get Temperature, pH, EC value of main tank |
| | | water. |
| | | Get flow meter output from water in flow meter |
| | | feeding flow meter and glycol cycle flow meter |
| Atmosphere | 1 | Get air temp and humidity |
| block | | Get CO2 concentration |
| LED Display | 1 | Display important data in the LED dot array |
| Board | | board. |

The Main Tank Block electronic are designed to perform the following and transmit the below data to control the system:
a. Collect the water level data through 3 level switches;
b. Get the water temperature through H2O Temperature probe.
c. Get the water pH value through pH probe.
d. Get EC value through EC probe.
e. Get how much water has been put into the machine through the water-in flow meter.
f. Get how much water has been fed to plants the machine through the feeding flow meter.
g. Feedback whether the glycol cycling is on by the glycol flow meter.
h. The water level limit switch will be on if the top water level sensor is on.

The Misting Tank Block electronic are designed to perform the following and transmit the below data to control the drain tank and misting tank.
a. Get the water level of the drain tank
b. Get the water level of the misting tank
c. Get the pH value of drain tank.
d. Get the EC value of drain tank.
e. Water level limit switch will be on when the high level sensor of the drain tank is on.
f. Get humidity and temperature of the root box
g. Get the O2 concentration of the root box
h. Control the piezo misters, fog fan and O2 solenoid.

The Atmosphere Control Board will collect the CO2 concentration, air temperature and humidity, then send that data to the center board through 485 bus. The center board will control the CO2 solenoid and AC system to maintain the CO2 concentration and air temperature at a level that will serve to optimize the plant growth within the plant growth system 100.

Referring again to FIG. 6, there is shown the pH sensor and EC sensor within the drain tank to measure and provide data as to what the nutrient levels are within the tank. The use of a pH sensor and EC sensor are exemplary and other sensors may be employed to provide specific data based upon on individual nutrient concentrations, the plant that is being grown, the stage of the growth cycle and the determination by the operator as to what they deem to be optimal. Thus, this can be a way of providing alternate chemical levels to study the effects on various plants at different stages of the growth cycle.

The sensors in the drain tank provide the operator with measurement data from both the mixing tank and the drain tank, such as water consumption, pH and electric conductivity. This information can be employed to calculate the relative uptake of nutrients and moisture and adjust the upcoming nutrient feed amounts accordingly. The compilation of a library of data permits developing standard feeding protocols that are customized for specific varieties of crop plants.

The operator can use the known compositions of the starting nutrient solution and the composition solution on the drain tank to calculate nutrient consumption. The system can also, record the amount of water being consumed by the plant and thus obtain, over time and on a real time basis, the comparative data to help determine actual grow programs/schedules that produce the best grow rates and yields.

In operation, the following exemplary parameters may be employed for the growing of Cannabis. It will be appreciated that these are only provided as indicia for the above species of plant and that the system may be advantageously employed with many other species of plants, both for growth, harvesting or for plant studies and experimentation. Thus, the parameters may be altered to provide optimal growth, harvesting or for plant studies and experimentation based upon the particular species within the system.

Exemplary Cannabis Parameters:

Air temperature
Shoot zone: 20-25° C. (68-77° F.)
Root zone: 18-22° C. (64-72° F.)
   Humidity—ambient and root box
Shoot zone: app. 60% during vegetative growth and 50% during flowering
Root zone: will be temporarily close to 100%, depending on the spray cycle.
   pH—main tank, drain box
pH control: main tank only (pH 5.8+/−0.1)
pH measurement: drain tank for feedback and main tank for adjustment
   Lights—spectrum, spread and intensity
Spectrum: full-spectrum LED, optimized for photosynthesis, with additional red:
Additional capacity to illuminate the plants temporarily with narrow-spectrum far red light of peak wavelength 730 nm but less than 10% of <700 nm for phytochrome conversion. Light intensity of 20-100 μmol×m·²×s⁻¹ PAR is sufficient. Can be achieved with app. 10 GU10 lights in one preferred embodiment of the plant growth system 100.
   Measurements: main tank and drain tank
Control: EC is controlled through nutrient feed. The measurements are used to adjust the nutrients and to monitor uptake in the root zone.
Misting and Fogging
   intermittent spraying or misting of the roots with 20-150 μM droplets.
   “dry” fog of 5 μM droplets is used for “shocking” roots in order to elicit biochemical responses and to adjust humidity in the root zone.
   fog is also used to increase humidity in the root zone to prevent drying as necessary.
   a particle filter may be advantageously employed to protect the nozzles
   alternatively, a temporarily increase in pressure may be employed for nozzle cleaning
O2—Range Determination
   The range for an ideal atmospheric O2 in the root zone for growth may be determined based upon the plants to be grown. Ideally, it should not drop below 20%, which is ambient but higher O2 might be beneficial. O2 content in the water can be adjusted by aeration and H₂O₂ addition, among other means.
   In order to reduce the risk of depleting oxygen in the root zone, it is recommended that the O2 is monitored and supplemented if necessary. Also, the higher CO2 level in the shoot zone might affect the root zone atmosphere.
   It is also recommended that the main tank be aerated.
CO2—range in the shoot zone
   CO2 in the shoot zone: 400 (ambient) to maximum 8,000 ppm (for pest control), maintained at 1000-2500 ppm throughout grow during the daytime and 400 ppm during the night.
   Use of pest control protocol (up to 8,000 ppm CO2) must be limited to necessity, as possibility of necrosis in the plants leaves from over exposure to CO2.
      Frequency of feeding/misting and fogging
Feeding: Typical intervals are 30 sec to 3 min spray with 30-240 min off, depending on the plant size and stage of development.
Fogging: The fog would normally be off and only come on for periods of up to 10 minutes with off cycles to be determined by the effect the treatment has on the plant and the necessity to not over-water the plant.
   Water quality requirements
Initially RIO reverse osmosis) water is used to ensure consistency of the nutrient solutions, avoid buildup of heavy metals, prevent scaling and establish baselines for growth
Subsequent to the establishment of baselines and determination of variations based upon nutrient/water concentrations and other mix variables:
   Obtain information about local water source from water department, including seasonal variations and establish critical parameters: hardness (Ca and total), alkalinity, pH, sodium, chloride, chlorine or chloramines, heavy metals
   verify with regular in-house and contract laboratory testing
   provide minimum filtration requirement: particles, activated carbon
   provide optional electronic wave pre-treatment for scale prevention and biofilm reduction
      Leaf movement
      Adequate air flow is essential to prevent mildew and ensure even environmental conditions. In the plant growth system 100 minimum air flow is determined by the cooling requirements and the intention to simulate natural air flow in an outdoors environment.
The A/C, airflow, and dehumidification systems should be independent.
During the night time cycle, when A/C units are not necessary, the humidity will be kept within parameter (<45% RH) with additional dehumidification.
   Day and night time frames
Lights are on 12-24 h. Cycles depend on the developmental stage of the plants.

Algorithms may be executed by a system-associated processor to optimize growth and energy consumption, track O2 movement, deliver/reclaim water, control all aspects of nutrition, utilize sensor data to control a system function, empirically determine a control sequence such as with a machine learning system, provide simulation-based control, determine and execute a nutrient schedule, such as one based on a condition such as nutrient deficiency.

Data from the system may be used in predictive analytics (e.g. Growth prediction), Growth cycle analysis, Event analysis (failure modes, Pathogen monitoring), performing a historical analysis of all controlled variables at rack level for entire growth cycle, perform growth modeling and statistics, generate computer simulation models (tool kit), and the like.

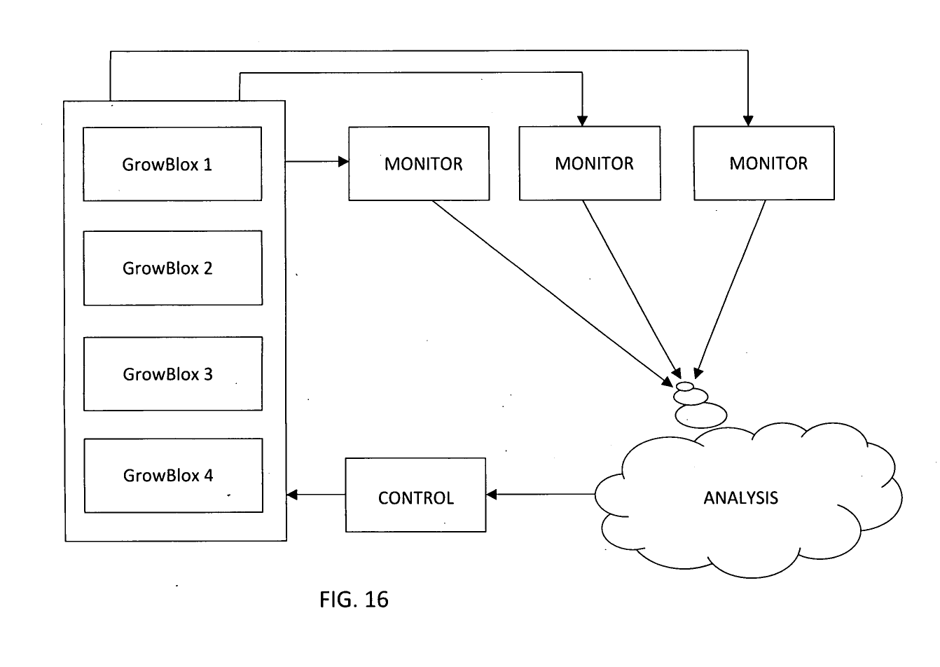

Referring to FIGS. 16 and 17, the integrated system data is monitored and provided to analysis module 1710 which can execute computer software, program codes, and/or instructions on a processor. The analysis module may also be provided with preclinical and clinical data from both peer-reviewed studies and anecdotal material to identify the most effective profiles of cannabinoids and terpenes for the treatment of conditions within targeted therapeutic categories.

As a further part of the analysis, the system then classifies existing cannabis strains containing the ideal ratios for treating specific diseases, or symptoms within specific targeted treatment categories by performing a preliminary cluster analysis of the active ingredient profiles of 30,000 Cannabis strains. This may be done in conjunction with a major testing laboratory to provide verifiable and ultimately certifiable data. It is a further part of the analysis and control system to identify optimum ratios of cannabinoids and terpenoids for the treatment of targeted disease categories and classify natural Cannabis strains that match the predicted ratios for the treatment of diseases in these targeted treatment categories.

It is yet a further part of the system to use patient validation through one or more software applications that are adapted for use with mobile devices such as a smartphone to validate the pre-selected strains or discover additional strains with defined cannabinoid/terpenoid ratios that are effective for the treatment of specific conditions. The patient validation is submitted through a GrowBLOX™—Patient Reported Outcome interface 1720 to a testing and trial drug determination engine 1730. The testing and trial drug determination engine 1730 allows for phase IV human clinical research by combining patient history data with real-time data collection and analysis of symptom surveys, cognitive tests, and biometric data to create, in real time, a personalized medical cannabis treatment program for each individual patient.

The long-term aggregated patient data sets provided to the Patient Reported Outcome interface 1720 and testing and trial drug determination engine 1730 strengthen the predictive treatment algorithms to improve future patient care. The analytical correlations between Cannabis strains (ratios of active ingredients) and symptom relief reach statistical significance to permit the determination that novel combinations of active ingredients are able to provide medical benefits in a repeatable and controlled manner for the targeted treatment categories.

The methods and systems described herein may be deployed in part or in whole through a machine that executes computer software, program codes, and/or instructions on a processor. The processor may be part of a server, cloud server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores.

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, social networks, and the like. Additionally, this coupling and/or connection may facilitate remote execution of program across the network. The networking of some or all of these devices may facilitate parallel processing of a program or method at one or more location without deviating from the scope of the disclosure. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs or codes as described herein and elsewhere may be executed by the client. In addition, other devices required for execution of methods as described in this application may be considered as a part of the infrastructure associated with the client.

The client may provide an interface to other devices including, without limitation, servers, cloud servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the disclosure. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, cloud servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements.

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, programs codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer to peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps thereof, may be realized in hardware, software or any combination of hardware and software suitable for a particular application. The hardware may include a general purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

The above systems and methods have been described in the context of an integrated incubation, cultivation and curing system and controls for optimizing and enhancing plant growth, development and performance of plant-based medical therapies it is to be understood that these systems and methods apply equally to methods and systems which employ soil to grow plants. Many of these systems and methods may incorporate soil into the racks holding the plants and also result in the benefits described for the systems and methods including, without limitation, the testing and trials engine and algorithms, applications and programs associated therewith.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

All documents referenced herein are hereby incorporated by reference.

What is claimed is:

1. An integrated plant cultivation system for optimizing, promoting and enhancing the rapid growth of a least one plant during one or more stages of its development cycle comprising:
a. at least one substantially closed tissue culture incubation chamber, into which at least one tissue culture receptacle is placed, said closed tissue culture incubation chamber having sensing means associated therewith to sense at least one of the light, temperature and/or humidity conditions within the tissue culture incubation chamber;
b. regulation means associated with the sensing means for regulating at least one of the light, temperature, nutrient and/or humidity conditions within the tissue culture incubation chamber to create at least one set of mini-clones from the tissue culture;
c. at least one substantially closed mini-clone growth chamber, into which at least one mini clone is placed, said closed mini-clone growth chamber having sensing means associated therewith to sense at least one of the light, temperature, nutrient and/or humidity conditions within the mini-clone growth chamber;
d. regulation means associated with the sensing means for regulating at least one of the light, temperature, nutrient and/or humidity conditions within the mini-clone growth chamber for such period as to permit the mini-clone to grow to a flowering plant;
e. at least one substantially closed growth chamber, into which at least one plant capable of flowering is placed, said closed growth chamber having at least one artificial growth inducing light source, at least one nutrient sensor adapted to determine, either directly or indirectly, the nutrient uptake of the plant, at least one environmental sensor adapted to determine, either directly or indirectly, atmospheric conditions within the substantially closed container and at least one growth sensor system adapted to determine, either directly or indirectly, the growth of the plant;
f. regulation means associated with the sensing means for regulating at least one of the light and/or atmospheric conditions within the growth chamber for such period as to permit the plant to flower;
g. a dispensing assembly containing at least one nutrient solution;
h. a misting assembly having a controllable interconnection to the dispensing assembly to provide a controlled amount of the nutrient solution into a controlled airflow;
i. a blower assembly in proximity to the misting assembly to create the controlled airflow from the misting assembly to the area of the root retention assembly;
j. a controller coupled to the artificial growth inducing light source and to the at least one growth sensor, environmental sensor and nutrient sensor adapted to: read information from the growth sensor to determine if growth has occurred; calculate the amount of nutrient to be delivered in the next feeding cycle; calculate the total number of on/off light cycles and a duration for each on/off cycle, and control the artificial growth inducing light source and alter the atmospheric conditions within the container to optimize the particular developmental cycle of growth desired; and,
k. a curing chamber having at least one group of cure controls for harvested flowers to control the temperature and humidity and permit normalization, standardization and consistency of the plants.

2. An integrated plant cultivation system in accordance with claim 1, wherein the plant is selected from a group of plants capable of providing plant-based medical solutions to combat at least one clinically diagnosed health issue.

3. A integrated plant cultivation system in accordance with claim 2 in which the plant is selected from a group consisting of plants from which may be derived medicinal extracts.

4. An integrated plant cultivation system in accordance with claim 3 in which the plant is selected from a group consisting of a species of Cannabis.

5. An integrated plant cultivation system in accordance with claim 2, wherein the system enhances the metabolic functions and the growing conditions of said plant by optimizing the nutrient absorption and provides variable nutrient supplies based upon developmental stage, physiological responses, absorption rates and/or other pre-established variables.

6. An integrated plant cultivation system in accordance with claim 2 in which the sensors provide data to a processor capable of administering the integrated plant cultivation system; a non-transitory memory configured to communicate with the processor, the non-transitory memory having instructions stored thereon; a monitoring module stored in the memory and operated by the processor, and configured to deliver an instruction to at least one of the regulation means based upon the data received, the monitoring module stored in the memory and operated by the processor, and configured to receive activity information associated with the plant; the monitoring module further configured to analyze the activity information based on criteria associated with the optimization to determine that an activity is an activity that optimizes at least one plant characteristic.

7. An integrated plant cultivation system in accordance with claim 6 in which the plant characteristics are selected from a group consisting of quality, purity, and/or consistency and the plant is selected from a group consisting of plants from which may be derived medicinal extracts.

8. An integrated plant cultivation system in accordance with claim 2 in which the nutrient and water solution is provided on a “just-in-time” basis.

9. An integrated plant cultivation system in accordance with claim 2 in which the at least one environmental sensor is monitored to determine atmospheric conditions and said conditions are altered to provide conditions that are pre-determined for optimal growth.

10. An integrated plant cultivation system in accordance with claim 2 in which the artificial growth inducing light source is varied to provide phytochrome modulation.

11. An integrated plant cultivation system in accordance with claim 9 in which the artificial growth inducing light source causes phytochrome modulation by providing far red-wavelength light.

12. An integrated plant cultivation system in accordance with claim 10 in which said phytochrome modulation produces a shortened cultivation cycle.

13. An integrated plant cultivation system for growing medicinal and recreational plants and non-medical plants comprising:
a. a tissue culture growth environment;
b. a nursery growth environment;
c. a growth environment in preparation for flowering;
d. a growth environment through flowering, each growth environment comprising one or more enclosures, a support structure positioned in the grow environment enclosure and adapted to support growing medicinal or recreational plants and non-medical plants;
e. sensors to monitor at least one real-time sensed parameter selected from a group consisting of temperature, light, humidity, carbon dioxide, pH level, water and/or nutrient delivery and/or misting schedules;
f. an nutrient delivery system coupled to the support structure and adapted to deliver micro-droplets of nutrient-laden mist or dry fog to the medicinal or recreational plants and non-medical plants;
g. a variable intensity and wavelength light system positioned in the grow environment enclosure and adapted for growing medicinal or recreational plants; and,
h. means for real time monitoring, managing and controlling the operation of the system based upon real-time sensed parameters.

14. An integrated plant cultivation system for growing medicinal and recreational plants and non-medical plants in accordance with claim 13 further comprising a system associated processor to execute an algorithm perform at least one of the following: (i) optimize growth/energy consumption; (ii) track O2 movement; (iii) deliver/reclaim water; (iv) handle all aspects of nutrition; (v) utilize sensor data to control a system function; (vi) iteratively determine a control sequence such as with a machine learning system; (vii) provide simulation-based control; or (viii) determine and execute a nutrient schedule, such as one based on a condition such as nutrient deficiency or one based on the developmental stage of the plant.

15. An integrated plant cultivation system in accordance with claim 14 further comprising a system associated processor to compile and analyze data from the system to generate predictive analytics, growth cycle analysis, event analysis, performing a historical analysis of all controlled variables at root and container level for an entire growth cycle, perform growth modeling and statistics, generate computer simulation models and provide optimization data for subsequent plant growth cycles.

16. An integrated plant cultivation system in accordance with claim 2 wherein the medicinal plants are produced in aseptic conditions.

17. An integrated plant cultivation system in accordance with claim 15 wherein the cultivation and processing protocols provide uniform medicinal extracts independent of the location of production, season or personnel.

18. An integrated plant cultivation system in accordance with claim 2 wherein the cultivation and processing further generate standardized propagation and cultivation conditions to provide uniform medicinal extracts independent of the location of production, season or personnel.

19. An integrated plant cultivation system in accordance with claim 2 wherein the medicinal plants are produced to provide plant extracts that are of reproducible chemical composition and purity.

20. An integrated plant cultivation system in accordance with claim 2 wherein all nutrients and water entering the grow chamber is recycled within the system and consumed by the plant, thus not generating any runoff.

21. An integrated plant cultivation system in accordance with claim 2 wherein programmed, temporary increase of atmospheric carbon dioxide concentration can be used to prevent or remove infestation by plant pest organisms.

22. An integrated plant cultivation system in accordance with claim 2 wherein each cultivation chamber comprises an enclosed, independent unit that can be programmed according to the need of the particular species, cultivar and developmental stage of the plant(s) in the unit, allowing for accommodating different crops in the same facility.
